(19) United States
(12) Patent Application Publication
(10) Pub. No.: US 2008/0319387 A1
(43) Pub. Date: Dec. 25, 2008

(54) Method and Apparatus for Inserting a Catheter Device

(76) Inventors: Shai Amisar, Tel-aviv (IL); Ronen Radomski, Haifa (IL)

Correspondence Address: NIXON & VANDERHYE, PC, 901 NORTH GLEBE ROAD, 11TH FLOOR, ARLINGTON, VA, 22203, US

(21) Appl. No.: 11/884,140

(22) PCT Filed: Feb. 14, 2006
(86) PCT No.: PCT/IL06/00194

§ 371 Date: Feb. 15, 2008

Related U.S. Application Data
(60) Provisional application No. 60/733,759, filed on Nov. 7, 2005.

(30) Foreign Application Priority Data
Feb. 14, 2005 (IL) 166863

(51) Int. Cl. A61M 25/092; A61M 25/09; A61M 25/18

(52) U.S. Cl. 604/95.04; 604/528; 604/533

(57) ABSTRACT

The present invention relates to systems and methods for inserting a catheter device into a subject, wherein the catheter insertion system generally comprises a flexible sleeve containing a catheter tube and advancing means for advancing the catheter contained within said system in a distal direction, wherein the flexible sleeve has a sealed proximal end and a distal end which may comprise a slender passage adapted to allow passage of said catheter tube therethrough while preventing backflow of fluid into the flexible sleeve.

### FIELD OF THE INVENTION

The present invention relates to intravascular catheter devices. More particularly, the invention relates to methods and apparatuses for facilitating and controlling the insertion of a catheter device.

### BACKGROUND OF THE INVENTION

It is known in the art to provide peripheral intravenous therapy using a catheter having a cannula or catheter tube to provide access into subcutaneous veins thereby to introduce medication, drugs, chemotherapy, nutrition and various other fluids into a vein of a subject. The present procedure includes inserting a hypodermic needle together with a catheter having an in-dwelling cannula into a suitable vein, withdrawing the needle and leaving the in-dwelling cannula in the vein. Such a catheter is typically provided with a suitable closure and various adapter mechanisms to enable the aseptic introduction of fluid medicaments from a hypodermic syringe or from an intravenous drip.

U.S. Pat. No. 5,478,326 describe an arterial device for the control of bleeding from a puncture in an artery wall. This device consists of a flexible cannula the size of which is gradually decreased from its access end towards its insertion tip, which enables bleed control by matching the partly inserted cannula to the size of the arterial puncture.

U.S. Pat. No. 3,757,771 describe a sterile inserter apparatus wherein an elongated catheter is adapted to move through the inserter via a sealed and flexible sleeve. The sealing sleeve is secured to a member that is connected to the inserter end after insertion of an in-dwelling cannula.

The catheter apparatus described in U.S. Pat. No. 3,825,001 includes catheter tubing sealed in a flexible plastic sheath having a male fitting secured to its distal end for connecting it to an inserter catheter and a female fitting secured to its proximal end. The male fitting is provided with axial openings to allow insertion of the catheter tubing through the inserter catheter.

An intravenous catheter system is described in WO 03/084428. This system comprises a multi-use entry-port element and a flexible catheter tube that is adapted for slidable insertion into a vein through the entry port. The flexible catheter tube is provided in a sterile environment containment element that allows insertion of the catheter tube in a generally non-sterile environment.

A considerable disadvantage of the prior art catheter devices stems from the requirement to insert the full length of the catheter tube into the artery site for performing continuous in vivo procedures. It is difficult to estimate the length of the catheter tube required for carrying out a specific procedure. Due to this requirement practitioners are often required to withdraw a partially inserted catheter tube whenever it is realized that a shorter catheter tube should be used.

None of the aforementioned publications provides means for controlling the insertion of the catheter. Heretofore, the insertion of intravenous catheter devices was carried our by grasping the catheter tube via the sterile containment and pushing the catheter tube inwardly into the entry port of the indwelling cannula. The prior art devices do not include control means for controlling the insertion of the catheter tube. In addition, the prior art catheter devices do not provide means for preventing accidental withdrawal, or means for preventing the use of excess force that may result in internal injuries. Moreover, the prior art devices do not provide means for carrying out the requisite procedure with a partially inserted catheter.

It is therefore an object of the present invention to provide methods and apparatuses for the controlled, aseptic insertion of an intravascular catheter into a peripheral blood vessel.

It is another object of the present invention to provide intravascular catheter insertion device comprising means for preventing accidental withdrawal of a partially inserted catheter tube.

It is a further object of the present invention to provide an intravascular catheter insertion device which permits the performance of in vivo procedures with a partially inserted catheter tube.

It is yet another object of the present invention to provide an intravascular catheter insertion device that can prevent exertion of excess forces during the insertion of the catheter tube.

It is still another object of the present invention to provide an intravascular catheter insertion device suitable for insertion of extremely thin catheter tubes, and that can prevent bleeding from the peripheral blood vessel into the catheter device.

It is also an object of the present invention to provide a method and accessories for trimming a catheter into a required length prior to its insertion in a sterile environment.

An additional object of the present invention is to provide disposable catheter insertion devices.

Other objects and advantages of the invention will become apparent as the description proceeds.

### SUMMARY OF THE INVENTION

The present invention provides a catheter insertion system that may be used for the controlled, aseptic insertion of catheters and similar indwelling tubular elements into the vasculature by means of the transcutaneous route.

The terms “resilient material”, “resilient section” and the like refer to materials, and elements made therefrom, that are capable of restoring their original shape and/or position after being compressed.

The present invention is primarily directed to a catheter insertion system comprising, at a first end, a distal entry port, at a second end a proximal section, a sealed catheter sleeve situated between said entry port and said proximal section, and further comprising advancing means for advancing a catheter contained within said system in a distal direction,
wherein said entry port comprises a hollow body terminating at one end in a hollow cannula suitable for insertion into a peripheral blood vessel and having an internal diameter suitable for permitting passage of an intravascular catheter, and terminating at the other end with a connector element;
wherein said proximal section comprises a hollow body having at least one external opening permitting the withdrawal and/or addition of fluid from or into the proximal opening of a catheter placed within said proximal section;
and wherein said sealed catheter sleeve is sealably connected at one of its ends to either said entry port connector element or to advancing means located between said sleeve and said entry port connector element, said sealed catheter sleeve being sealably connected at its other end to said proximal section;
such that when the aforementioned elements are connected together, a continuous hollow passageway exists from said proximal section through to the terminal portion of said hollow cannula, wherein said passageway contains the catheter that is to be inserted into a peripheral blood vessel.

Optionally, the aforementioned entry port may also comprise an external opening permitting the withdrawal and/or addition of fluid from or into the proximal opening of a catheter placed within said entry port.

It should be noted that the term “proximal” is used here to refer to elements of the catheter device which are located in relative proximity to the operator, and the term, “distal” is used herein to refer to elements of the catheter device the location of which is relatively distant from the operator.

In one preferred embodiment of the aforementioned catheter insertion system, the advancing means comprises the sealed catheter sleeve and a plurality of blades situated in close proximity to the distal end of said sleeve, such that when said sleeve is grasped and advanced in a distal direction, the catheter situated within said sleeve is similarly advanced, and said sleeve is progressively cut by said blades, thereby facilitating its removal from said system.

In another preferred embodiment of the catheter insertion system of the present invention, the advancing means comprises a flexible tube situated between the entry port and the sealed catheter sleeve, wherein said tube is adapted to permit an operator to grasp and compress said tube such that the catheter situated therewithin may be advanced in a distal direction. In a particularly preferred embodiment, the flexible tube contains corrugations along at least part of its length, such that said corrugations provide increased friction between the operator's hand and the catheter tube.

In accordance with a further preferred embodiment of the catheter system of the present invention, the advancing means comprises a rotating wheel mechanism situated between the entry port and the sealed catheter sleeve, such that rotation of an externally-situated thumb wheel causes rotation of one or more internally-situated wheels that cause the catheter to move in a distal or proximal direction. The internally-situated wheels are preferably designed to provide a limited amount of friction between said wheels and said catheter tube, thereby limiting the amount of force applied on the thumb wheel.

In a further preferred embodiment, the advancing means comprises a rotating wheel mechanism as disclosed hereinabove and described in further detail hereinbelow, and the sealed catheter sleeve consists of a pair of mutually adhered sealing strips, wherein said rotating wheel mechanism further comprises a pair of winding wheels for separating and winding each of said sealing strips onto a spool.

In another preferred embodiment of the system of the present invention, an affixing device is utilized for affixing the proximal section of the system to the distal section for allowing performance of procedures with a partially inserted catheter. The affixing device preferably comprise fastening arms adapted to clasp the distal section and retaining arms adapted to receive and hold the proximal section of the system. The affixing device may further comprise supporting pins for wrapping a portion of the adhered sealing strips therebetween, whenever the length of the none-inserted strip bilayer is greater than the length of the affixing device.

In another preferred embodiment of the system of the present invention, a sealed separator is utilized for separating the adhered sealing strips, exposing the catheter sealed therein, and advancing it into the entry port. The separator comprises a proximal aperture and retaining members for guiding the catheter sealed in the adhered strips, entering the separator via said proximal aperture, towards an opening of a passage tube linked to the entry port. The adhered strips are separated in the vicinity of the passage tube opening, advanced along the sides of splitting means, and leave the separator via apertures provided thereon. The catheter is exposed and advanced into said passage tube and therefrom into the entry port. The tips of the separated strips may be adhered outside the separator to allow convenient insertion of the catheter by pulling the adhered tips distally.

In another aspect, the present invention is also directed to an advancing mechanism for advancing a sterile catheter in a distal direction, wherein said mechanism comprises a sealed spool containing a sterilized catheter wound around a central axis in a spiral manner, and a distal exit, such that said catheter is rolled in or out via said distal exit by rotations of the spool. The distal exit is connected to an entry port via suitable connector means having an inner passage for advancing the catheter therethrough. The inner lumen of the catheter can be accessed via a catheter port connected to the proximal end of the catheter wound, allowing performance of procedures with a partially inserted catheter. After insertion of the catheter tube the spool is dismantled and a portion of the catheter tube and the catheter port attached thereto are affixed to the arm of the treated patient.

Optionally, the sealed spool containing the sterilized catheter wound is advanced utilizing the advancing means comprising a rotating wheel mechanism situated between the entry port and the sealed spool, such that rotation of the externally-situated thumb wheel causes rotation of one or more internally-situated wheels that cause the catheter to move in a distal or proximal direction. The internally-situated wheels are preferably designed to provide a limited amount of friction between said wheels and said catheter tube, thereby limiting the amount of force applied on the thumb wheel.

According to yet another preferred embodiment the present invention is directed to a catheter insertion system comprising a flexible sleeve containing a catheter tube, wherein the flexible sleeve has a sealed proximal end, and wherein the distal end of the flexible sleeve comprises a slender passage adapted to allow passage of the catheter tube therethrough while preventing backflow of fluid into the sleeve.

The catheter insertion system may further comprise a resilient portion formed near the slender passage. This resilient portion may be implemented as a corrugated portion of the flexible sleeve.

The catheter insertion system may further comprise tear lines longitudinally passing along at least a portion of the length of the flexible sleeve, for facilitating tearing thereof.

The catheter insertion system may further comprise one or more permeable portions provided on the flexible sleeve for allowing gas sterilization of the interior of the flexible sleeve. Alternatively or additionally, the proximal end of the flexible sleeve is sealed by a plug having a bore, wherein one end of the bore opens into the interior of the flexible sleeve, and wherein the other end of the bore is covered by a permeable sheet for allowing gas sterilization of the interior of the flexible sleeve.

Preferably, the proximal end of the flexible sleeve is closed by a guide wire stopper element, wherein the guide wire stopper element comprises an aperture suitable for the passage of a guide wire holder connected to the proximal end of a guide wire, and wherein the passage of the guide wire holder into the flexible sleeve can be prevented by rotation of the guide wire holder about the axis of the guide wire.

Optionally, a fastening cap adapted to fit in or over the proximal side of the guide wire stopper element is used for sealing the flexible sleeve, wherein the fastening cap may comprise gripping means adapted to grip the guide wire holder and prevent passage thereof into the flexible sleeve.

The slender passage element may be a part of a splittable head sealably fitted into the distal end of the flexible sleeve, wherein the splittable head is constructed such that it can be longitudinally split into two or more parts, thereby facilitating tearing of the flexible sleeve. Additionally, the splittable head may comprise pins adapted to be engaged in respective apertures provided in the flexible sleeve. The splittable head may further comprise splitting tabs provided on each splittable part thereof.

The catheter tube may comprise a lateral port adapted to allow access to the lumen of the catheter tube by a syringe needle passing through the wall of the flexible sleeve. Additionally, the flexible sleeve may also comprise a lateral port adapted to allow sealable access to the interior of the flexible sleeve by a syringe needle.

The proximal end of the catheter insertion device may be sealed by a sealing plug comprising a slender passage through which the catheter tube is sealably passed such that its proximal port is external to the sleeve.

The catheter insertion system may further comprise a depressible element adapted for advancing the catheter tube contained in the flexible sleeve, wherein the depressible element comprises a flexible inverted “VI” structure that is capable of advancing the catheter tube distally when pressed.

The present invention is also directed to an adapter for a catheter tube cutter, comprising a hollow connector for connecting a catheter insertion device thereto, a base connected to the hollow connector, and an arm connected to the base opposite to the hollow connector, the arm comprises an aperture inline with an opening of the hollow connector, wherein a gap formed between the connector and the arm is suitable for fitting a catheter tube cutter therein and passing a catheter tube in the hollow interior of the connector via a cutter passage provided in the catheter cutter and the aperture in the arm.

### BRIEF DESCRIPTION OF THE DRAWINGS

The present invention is illustrated by way of example in the figures of the accompanying drawings, in which like references indicate similar elements and in which:

FIG. 1A is a perspective view of a catheter insertion device of the invention comprising a firm sealing sleeve and small blades;

FIG. 1B is a longitudinal section view of the catheter insertion device shown in FIG. 1A;

FIG. 1C is a longitudinal section view of the distal portions of the catheter insertion device shown in FIG. 1A;

FIG. 1D is a longitudinal perspective section view of the catheter insertion device shown in FIG. 1A in its “inserted” state;

FIG. 2A is a perspective view of a catheter insertion device of the invention comprising a flexible corrugated tube;

FIG. 2B is a longitudinal section view of the catheter insertion device shown in FIG. 2A;

FIG. 2C is a longitudinal section view of the distal portions of the catheter insertion device shown in FIG. 2A;

FIG. 2D is a perspective longitudinal section view of the catheter insertion device shown in FIG. 2A in its “inserted” state;

FIG. 3A is a perspective view of a catheter insertion device of the invention which comprises a rotating mechanism for advancing the catheter tube;

FIG. 3B is a longitudinal section view of the distal portions of the catheter insertion device shown in FIG. 3A;

FIG. 3C is a longitudinal section view of the catheter insertion device shown in FIG. 3A;

FIG. 3D shows the elements inside the advancing mechanism used in the catheter insertion device shown in FIG. 3A;

FIG. 3E is a perspective view of the catheter insertion device shown in FIG. 3A in its “inserted” state;

FIG. 4A is a perspective view of a catheter insertion device of the invention which comprises a stripe for sealing and advancing the catheter tube;

FIG. 4B is a longitudinal section view of the distal portions of the catheter insertion device shown in FIG. 4A;

FIG. 4C is a longitudinal section view of the catheter insertion device shown in FIG. 4A;

FIG. 4D is a bottom close-up on the inner elements of the advancing mechanism shown in FIG. 4A;

FIG. 4E is a top close-up view on the inner elements of the advancing mechanism shown in FIG. 4A;

FIG. 4F is a perspective view of a catheter insertion device of the invention affixed in a partially inserted state using an affixing device;

FIG. 5A is a perspective view a catheter insertion device of the invention wherein the catheter tube is advanced by a “pull” action;

FIG. 5B is a top angled view of the catheter insertion device shown in FIG. 5A;

FIG. 5C is a longitudinal section view of the distal portions of the catheter insertion device shown in FIG. 5A;

FIG. 5D is a perspective view of the catheter insertion device shown in FIG. 5A in its “inserted” state;

FIG. 6 shows an advancing mechanism wherein the catheter tube is wound on a spool;

FIG. 7A is a perspective view of another catheter insertion device of the invention consisting of a single flexible sleeve;

FIG. 7B is a close-up perspective view on the proximal portion of the catheter insertion device shown in FIG. 7A;

FIG. 7C is a longitudinal section view of the distal portion of a tearable application of the catheter insertion assembly shown in FIG. 7A;

FIG. 7D is a perspective view of the tearable application shown in FIG. 7C;

FIG. 7E demonstrates tearing the flexible sleeve of a tearable catheter insertion device;

FIG. 8A schematically illustrates a conventional catheter cutter;

FIG. 8B schematically illustrates a cutter adapter according to the invention;

FIG. 8C schematically illustrates fitting a conventional catheter cutter in the cutter adapter of the invention;

FIG. 8D is a perspective view of a further catheter insertion device of the invention based on a flexible corrugated tube that is connected to a catheter cutter device;

FIG. 8E is a close-up on the distal portion of the assembly shown in FIG. 8D;

FIG. 8F is a close-up on the rear portion of the catheter insertion device shown in FIG. 8A which comprises a guide-wire stopper mechanism;

FIG. 8G is a longitudinal section of the rear portion shown in FIG. 8F;

FIG. 8H is a close-up on the guide wire stopper mechanism shown in FIG. 8F;

FIG. 9A illustrates a guide-wire stopper mechanism comprising a fastening cap;

FIG. 9B is a perspective view of the guide-wire stopper mechanism with the fastening cap;

FIG. 9C is a perspective view of the fastening cap;

FIG. 9D is a rear view of the guide-wire stopper mechanism shown in FIG. 9A;

FIG. 9E is a perspective front view of the fastening cap;

FIG. 10A is a perspective view of a catheter insertion device of the invention having splittable head;

FIG. 10B is a longitudinal view of the splittable head of the catheter insertion device shown in FIG. 10A;

FIG. 10C is a longitudinal view of a splitted portion of the splittable head shown in FIG. 10B;

FIG. 10D schematically illustrates splitting of the splittable head and flexible sleeve of the catheter insertion device shown in FIG. 10A;

FIG. 11A is a longitudinal section view of a catheter insertion device of the invention which comprises a depressible element;

FIG. 11B is a top view of the catheter insertion device shown in FIG. 11A;

FIG. 11C is a perspective view of the depressible element of the catheter device shown in FIGS. 11A and 11B;

FIG. 12 schematically illustrates a catheter tube comprising a slidable element comprising an antimicrobial substance;

FIG. 13A schematically illustrates a guide wire insertion device and a catheter insertion device having a splittable head wherein the catheter port exits the device via the rear end of the device;

FIG. 13B demonstrates insertion of the guide wire into the catheter when connecting the devices shown in FIG. 13B;

FIG. 13C schematically illustrates devices similar to those shown in FIGS. 13B and 13C wherein the catheter tube is threaded over a guide wire contained in the catheter insertion device;

FIG. 14A schematically illustrates a catheter insertion device of the invention wherein the catheter tube is provided with a lateral port; and

FIG. 14B schematically illustrates a catheter insertion device of the invention wherein the catheter tube and the flexible sleeve are provided with a lateral port.

### DETAILED DESCRIPTION OF PREFERRED EMBODIMENTS

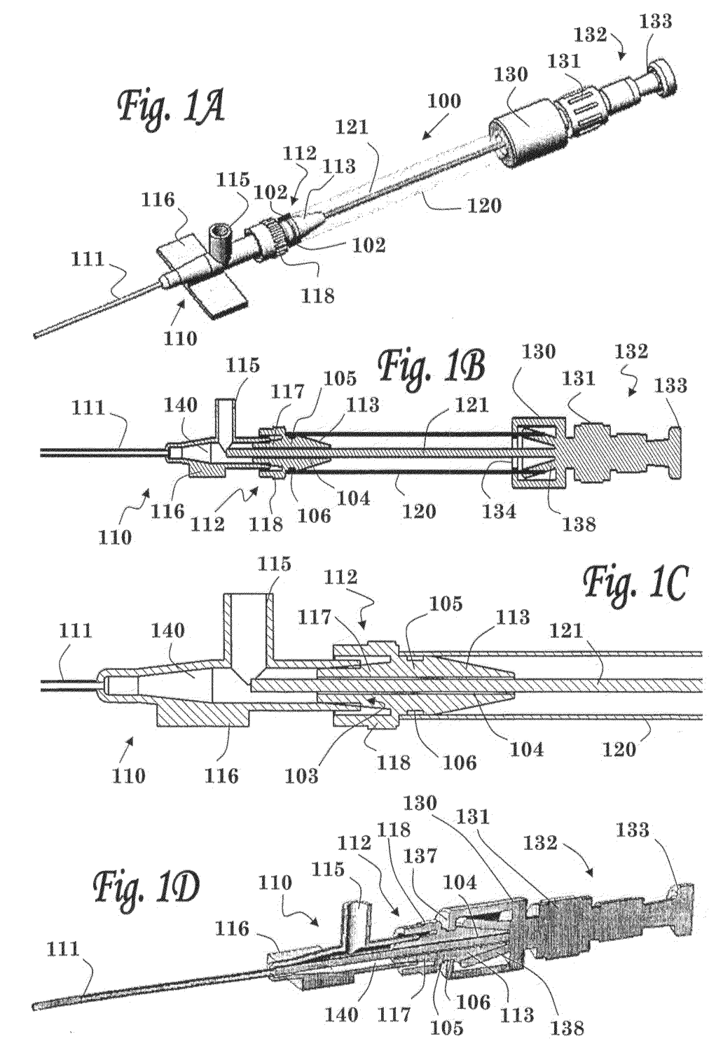

FIG. 1A shows a perspective view of a catheter insertion device of the present invention (before insertion of the catheter tube into the blood vessel). In this preferred embodiment the intravascular catheter insertion device 100 comprises three main elements: entry port 110 (bearing an insertion cannula 111 at its distal-most end), proximal hub 132 and sealing sleeve 120, said sleeve 120 being located between said entry port 110 and said proximal hub 132. Sealing sleeve 120 is sealably connected at its proximal end to the inner bore of proximal hub 132, and sealably connected at its distal end to entry port 110, as will be described in more detail hereinbelow.

Before use, the device is preferably provided in two sections: (i) a distal section comprising the entry port 110 (having a disposable needle inserted within its central bore), and (ii) a proximal section comprising proximal hub 132 connected to sealing sleeve 120.

Following placement of the insertion cannula 111 (which forms part of entry port 110) into the desired blood vessel (not shown), the aforementioned proximal section is connected to the distal section, such that the catheter insertion device comprises a sealed, internal passageway extending from proximal hub 132, through sealing sleeve 120 to the distal extremity of entry port 110. As best seen in the longitudinal view of FIG. 1B, catheter tube 121 is disposed within said internal passageway. During use of the insertion device, said catheter 121 is advanced through the internal passageway, in a distal direction, such that it protrudes from the distal end of entry port 110 (through the internal bore of insertion cannula 111), thereby becoming inserted into the desired blood vessel. Each of the three main structural elements defined above will now be described in more detail.

As best seen in FIG. 1C, the most distally placed of the three main structural elements of the device, namely entry port 110 comprises a hollow body (preferably of round cross-sectional geometry) which continues distally as hollow insertion cannula 111, and ends proximally with a connector 112. Optionally, the main body of entry port 110 also contains one or more hubs 115 that permit the introduction or removal of fluids into or from the internal bore of the entry port. Entry port 110 further comprises flexible wings 116 which may be taped or otherwise secured to the skin surface of the patient being treated, thereby stabilizing the entire catheter insertion device 100. The proximal extremity of entry port 110 is adapted to reversibly connect with connector 112, which is located at the distal end of sealing sleeve 120, as will be described in due course. Before use, entry port 110 is fitted with an appropriately-sized disposable needle (not shown) within its internal bore, such that the tip of said needle projects distally from the end of insertion cannula 111. By means of said needle, said cannula may be inserted into the desired blood vessel (subcutaneous over-the-needle cannula insertion). The needle is then removed from entry port 110, and the proximal end of said entry port is then connected with sealing sleeve 120 by means of connector 112, the structure of which will be described in more detail hereinbelow.

The opening of the hollow insertion cannula 111 inside entry port 110 may comprise a rubber septum (not shown), having an annular (“O”) or star shape, that applies some pressure (low tack) to the outer surface of catheter tube 121 in order to prevent blood from flowing via entry port 110 into sleeve 120.

Turning now to the most proximally-located element, as shown in FIGS. 1B and 1C the proximal hub 132 comprises a socket 130, for receiving the proximal end of catheter 121 and a quick connector (e.g., Luer lock connector) 131 for connecting the guide wire assembly 133 (guide wire holder) to proximal hub 132, as provided in standard Peripherally Inserted Central Catheters (PICC).

Quick Connector 131 can be unscrewed, thereby exposing the Luer opening and providing access to the proximal opening of catheter tube 121. Quick connector 131 and guide wire holder 133 are usually discarded after the insertion of the catheter tube is completed.

Sealing sleeve 120 is preferably made of a type of transparent plastic silicon having thick walls for providing rigidity to the catheter insertion device 100, and is sealably attached at its proximal end to proximal hub 132, as described hereinabove. The distal end of sleeve 120 is fitted with a connector 112, which is used to sealably connect the sleeve with entry port 110. Projecting in a proximal direction from the external surface of connector 112 are two or more small blades 102, which are disposed such that they are covered by sealing sleeve 120, with their cutting edges facing in an outward direction. By means of this arrangement, small blades 102 are used to incise sealing sleeve 120, thus enabling its removal during the catheter insertion process, as will be described in more detail hereinafter.

Referring now to the longitudinal section view shown in FIG. 1C the structure of connector 112 and the interaction between said connector and entry port 110 will be described in more detail. In this example, connector 112 comprises a hollow cylindrical member 118, adapters 113 and 117, and an axial bore 104 passing therebetween. Adapter 117 extends outwardly from the interior of connector 112. Adapter 117 is received in the proximal opening 103 of entry port 110 and thereby provides a sealed connection by fitting the inner surface of proximal opening 103 over the tapering tip of adapter 117. This connection provides a sealed and secure connection between the central bore 140 of entry port 110 and axial bore 104 of connector 112.

A cylindrical section 105 is provided between adapter 113 and the hollow cylindrical member 118 of connector 112. Cylindrical section 105 is adapted to be received into one end of the sealing sleeve 120 by fitting the inner surface of the sleeve's end around it. In this way the catheter tube 121 can be inserted via the sterile zone defined by the passage provided through sealing sleeve 120, axial bore 104, central bore 140, and therefrom into the insertion cannula 111. Annular groove 106 provided on the outer surface of cylindrical section 105 is used to receive socket 130 in a secure and sealed fashion.

The catheter insertion device, as described hereinabove is used in the following manner.

Firstly, insertion cannula 111 together with its' indwelling disposable needle (not shown) is inserted into the desired blood vessel, following puncture of the overlying skin and the wall of said vessel by the tip of said needle. The entry port is then secured in place by affixing flexible wings 116 to the skin surface. Next, the needle is removed from entry port 110, and the proximal portion of the device (i.e. sleeve 120 pre-connected to proximal hub 132 is then connected to the proximal end of entry port 110 by means of connector 112. The catheter tube 121 is then slidably inserted through the insertion cannula 111 into the desired blood vessel by advancing proximal hub 132 in a distal direction, i.e. towards connector 112. In this way portions of catheter tube 121 are inserted into the insertion cannula 111 and the sealing sleeve 120 is pressed against blades 102, which “peel off” portions of said sleeve.

The proximal hub 132 is thus directed distally until the entire sleeve is peeled off and socket 130 receives adapter 113 into its hollow interior, as shown in FIG. 1D. The interior of socket 130 comprise a female fitting 138 adapted to fasten the proximal end of sleeve 120 inside socket 130, and to receive adapter 113 and provide a sealed connection between the axial bore 104 of connector 112 and the inner bore (not shown) of proximal hub 132. Aperture 134 (FIG. 1B) at the distal side of socket 130 define an inward annular protrusion 137 that is engaged with the annular groove 106 when the insertion of the catheter tube 121 is completed, thereby providing a sealed connection with connector 112.

The length of proximal hub 132 is generally in the range of 10 to 35 millimeters, and preferably about 20 millimeters, and the diameter of its inner bore is generally slightly larger than the external diameter of catheter tube 121. The length of adapter 130 is generally in the range of 10 to 30 millimeters, and preferably about 20 millimeters, its outer diameter is generally in the range of 8 to 20 millimeters, and preferably about 14 millimeters, and the sizes of its aperture and female fitting 138 provided therein should be defined according to the dimensions of adapter 113. Proximal hub 132 can be fabricated from any suitable material (e.g., metal, plastic), and utilizing fabrication techniques, such as ordinarily use in the fabrication of such devices (e.g. injection molding). The length of catheter tube 121 is generally in the range of 150 to 700 millimeters, and preferably about 250 millimeters, and it diameter is generally in the range of 0.8 to 2 millimeters, and preferably about 1 millimeters. Catheter tube 121 can be fabricated from any material suitable for catheters implementations such as polyurethane, silicone, or Teflon.

The length of the sealing sleeve 120 should be determined according to the length of the catheter tube used, and its diameter is generally in the range of 10 to 70 millimeters, and preferably about 25 millimeters. The sealing sleeve 120 is preferably transparent and may be fabricated from Silicone or polyurethane. The thickness of the walls of the sealing sleeve is generally in the range of 0.1 to 2 millimeters, and preferably about 1 millimeter, in order to obtain some rigidity of said sleeve. The length of connector 112 is generally in the range of 5 to 15 millimeters, and preferably about 10 millimeters, its outer diameter is generally in the range of 5 to 15 millimeters, and preferably about 8 millimeters, and its inner diameter is preferably defined according to the parameters of the entry port to be used.

Connector 112 is preferably fabricated by injection molding techniques from polypropylene, polyethylene, or ABS, materials. The Blades 102 are preferably made from a thin, sharp metal strip the length of which is generally in the range of 1 to 8 millimeters, and preferably about 2 millimeters.

As described hereinabove the catheter insertion device of the invention generally consists of three portions: a proximal portion comprising the catheter port, a guide wire grip, and a socket for sealably connecting said portion to the distal portion; an intermediate portion comprising a sealed containment including portion of the catheter tube sealed therein, and a distal portion connected to the entry port and comprising means for removing said sealed containment and advancing the portions of exposed catheter tube therethrough into the entry port. In general, the elements of the proximal portion, and the entry port, in the different embodiments of the catheter insertion device of the invention are similar in structure and shape, mutatis mutandis, and therefore they will not be described in details hereinafter. Moreover, it should be understood that the subcutaneous “over the needle” insertion of the cannula described hereinbefore is similarly performed in all other embodiments of the invention, and therefore will not be described with reference to each embodiment of the invention for the sake of brevity.

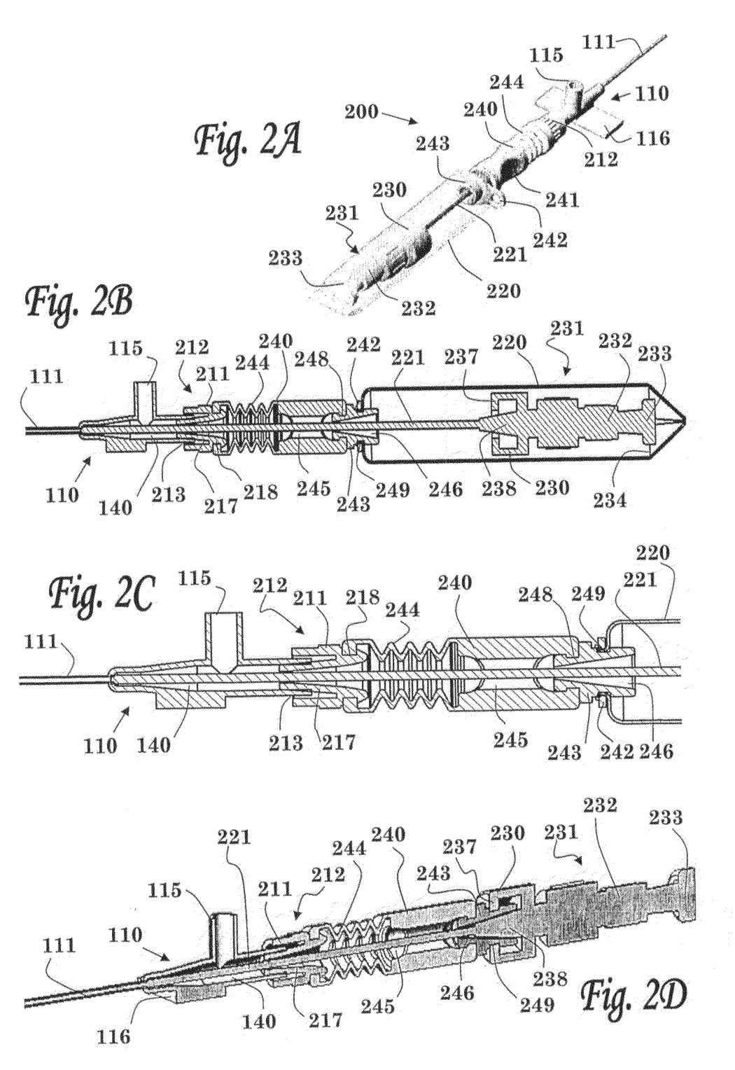

Another catheter insertion device 200 of the invention is shown in FIGS. 2A-2D. FIGS. 2A-2C shows the catheter insertion device 200 prior to its insertion into a blood vessel. This embodiment comprises a sealing sleeve 220 enclosing a proximal hub 232 and a catheter tube 221 disposed therewithin. The distal end of the sealing sleeve 220 is secured to the proximal side of adapter 243 by a clip (e.g., “U” shaped clip) 242, and the distal side of adapter 243 is attached to the proximal end of a flexible and partially corrugated tube 240. Adapter 243 connects the interiors of sealing sleeve 220 and flexible tube 240 via inner bore 246 (FIG. 2B) formed therewithin.

Proximal hub 232 comprise a socket 230 a quick connector 231 (e.g., Luer lock), and a guide wire holder 233. The catheter tube 221 passes via the interior of proximal hub 232 and its lumen can be accessed via port 233. Proximal hub 232 further comprises a sleeve clamp 234. The sealing sleeve 220 includes a single opening at the distal end thereof, and the proximal end of sleeve 220 is permanently sealed. The entry port 110 comprises an insertion cannula 111, hub 115, and wings 116 for securing entry port 110 to the body of a treated subject.

Depressions 241 (FIG. 2A), on lateral sides of flexible tube 240 and adjacent to its proximal end, allows the practitioner to conveniently grasp the lateral sides of catheter tube 221 passing via the interior space 245 (FIG. 2B) of tube 240. The section adjacent to the distal end of flexible tube 240 comprise annular corrugations 244 for advancing the catheter tube 221 passing therewithin by grasping its lateral sides via depressions 241 and advancing the same in a distal direction with the entire proximal section of the catheter insertion device 200 by applying a forward pressure and thereby reducing the total length of corrugated section 244. Corrugated section 244 serves as a spring for restoring the original length of flexible tube 240, thereby allowing insertion of portions of the catheter tube 221 via forward movements without requiring application of backward movements before insertion of further portions of the catheter 221. Similar operation can be achieved by using other resilient mechanisms, such us springs and/or resilient tubes, or combinations hereof.

FIG. 2C shows a longitudinal section view of the distal portion of catheter insertion device 200 comprising flexible tube 240, connector 212, and adapter 243. Catheter tube 221 is shown passing through passage 246 provided inside adapter 243, via the interior space 245 of flexible tube 240, via conical passage 217 inside connector 212, and therefrom into the central bore 140 of entry port 110. The tip of conical passage 217 preferably tightly fits over the outer surface of catheter tube 221 in order to prevent blood from flowing via entry port 110 into flexible tube 240.

Adapter 243 comprises cylindrical sections at its proximal and distal sides, wherein the cylindrical section at the distal side comprise annular groove 248 that receives an inward annular protrusion at the proximal end of flexible tube 240, and wherein the cylindrical section at the proximal side comprise an annular groove 249 adapted to receive the distal end of sealing sleeve 220, which is securely sealed thereto by clip 242, and an inward annular protrusion 137 at the distal side of socket 230. Optionally, an additional annular groove may be provided proximally adjacent to groove 249 for receiving the annular protrusion of socket 230. In this way a sealed passage is defined for advancing the catheter tube 221 starting from the interior of sleeve 220 via the interior space 245 of flexible tube 240 and through the central bore 140 of entry port 110.

Connector 212 consists of a cylindrical hollow member 211 having an opening 213 at a distal side thereof, wherein the diameter of opening 213 is greater than the outer diameter of the proximal end of entry port 110, thereby allowing insertion of the proximal end of entry port 110 into the interior of connector 212. The conical passage 217 inside connector 212 extends outwardly via opening 213, such that its distal end leaves the interior of cylindrical hollow member 211. The diameter of the distal end of conical passage 217 is smaller than the diameter of the opening at the proximal end of entry port 110 thereby providing secure sealing therebetween by fitting the proximal end of entry port 110 a round the distal end of conical passage 217. Connector 212 comprises a cylindrical section at its proximal side, where said cylindrical section includes an annular groove 218 which receives the inward annular protrusion 137 at the distal end of flexible tube 240.

In this preferred embodiment rigidity of the sealing sleeve 220 is not required, such that it may be produced from a type of transparent plastic silicon or from Polyethylene. The catheter tube 221 is slidably inserted via the insertion cannula 111 into the blood vessel by grasping its lateral sides via depressions 241 and forwardly pushing it toward entry port 110, thereby reducing the total length of the corrugated section 244 and moving portions of catheter tube 221 towards the insertion cannula 111. The inner diameter of conical passage 217 at its distal tip approaches the outer diameter of catheter tube 221 for tightly fitting it around the outer surface of catheter 221, in order to provide a grip of catheter tube 221 for preventing its withdrawal after the applied grasp of depressions 241 is released. When said grasp is released, the corrugated section 244 expands backwardly and restores its previous shape, thereby sliding depressions 241 and adapter 243 backwardly over catheter tube 221.

By repeatedly pushing portions of the catheter tube 221 the Proximal hub 232 is moved forwardly until the socket 230 receives the cylindrical section at the proximal side of adapter 243 inside its hollow interior, as shown in FIG. 2D. Male fitting 238 inside socket 230 is adapted to be received in passage 246 and thereby provide a sealed connection between the interior space 245 of tube 240 and the inner bore (not shown) of proximal hub 232.

At this stage inward annular protrusion 237 at the distal side of socket 230 is engaged with annular groove 249 which provides a sealed and secured connection therebetween. After the secured connection between adapter 243 and socket 230 is obtained the sealing sleeve 220 can be removed by detaching clip 242 from annular groove 249. After the sealing sleeve 220 is removed the requisite procedure can be carried out.

The length of adapter 243 is generally in the range of 8 to 20 millimeters, and preferably about 12 millimeters. Its diameter is preferably in the range of 5 to 15 millimeters, and preferably about 8 millimeters. Adapter 243 is preferably made of polypropylene, polyethylene, or Teflon. The length of the cylindrical sections at the proximal and distal sides of adapter 243 is generally in the range of 4 to 12 millimeters, and preferably about 8 millimeters, and the diameter of annular groove 249 is determined according to the diameter of the sealing sleeve for providing sealed connection therewith, and the diameter of annular groove 248 is determined to provide sealed closing according to the diameter of flexible tube 240.

Flexible tube 240 is preferably made of flexible, “memory” type of material (i.e., capable of restoring its original shape) such as Silicone, and manufactured utilizing known techniques, such as injection molding. Its length is generally in the range of 15 to 40 millimeters, and preferably about 25 millimeters, and its diameter is generally in the range of 8 to 20 millimeters, and preferably about 15 millimeters. The dimensions of connector 212 are generally similar to the dimensions of connector 112, and accordingly the dimensions of the conical passage 217 should be determined according to the entry port 110 used. Clip 242 is preferably made from a type of flexible hard material such as polypropylene, or stainless steel.

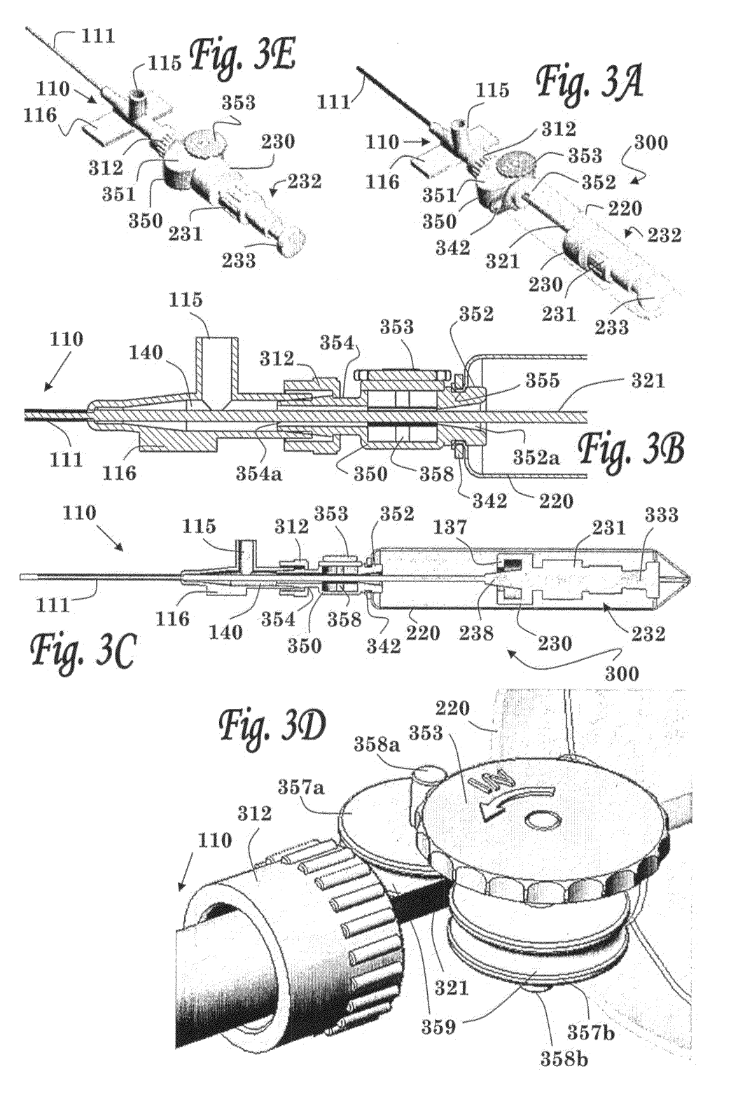

An advancing mechanism 350 can be used for advancing the catheter tube 321, as shown in FIGS. 3A to 3D. The catheter insertion device 300 in this embodiment comprises a proximal hub 232 and catheter tube 231 enclosed within sealing sleeve 220, advancing mechanism 350, and a hollow connector 312 connecting the interior of advancing mechanism 350 with the central bore (140 shown in FIG. 1C) of entry port 110. The opening at the distal end of the sleeve 220 is sealed by clip 342 placed over its edge and fastening it over an annular groove 355 (FIG. 3B) provided at the proximal end of advancing mechanism 350. Advancing mechanism 350 comprises cylindrical sections, 352 (comprising annular groove 355)—at its proximal end and 354—at its distal end, a sealing cover 351 (FIG. 1), and an applicator wheel 353. Axial bores, 352a and 354a, (FIG. 3B) are provided inside cylindrical sections 352 and 354, respectively. Cylindrical section 352 is adapted to be received inside socket 230 and comprises an annular groove adapted to receive clip 342 while grasping the edge of sleeve 220 over it. Said annular groove is also adapted to receive an inward annular protrusion 137 at the distal side of socket 230. Alternatively, an additional annular groove may be provided for receiving said inward annular protrusion 137 at the distal side of socket 330.

As shown in FIG. 3B connector 312 comprises an inward annular protrusion at its proximal side adapted to fit over the outer surface of cylindrical section 354. The proximal opening of entry port 110 is adapted to fit over the distal potion of cylindrical section 354 and thereby to provide a sealed connection between the central bore 140 of entry port 110 and the internal space of advancing mechanism 350. Connector 312 may be secured to the proximal end of entry port 110, for example via an inward annular protrusion (not shown) provided at the opening at its distal, where said annular protrusion is adapted to fit over the outer surface of the proximal end of entry port 110. In this way a sealed passage is provided for advancing catheter tube 321 from the sealing sleeve 220 via advancing mechanism 350 utilizing the axial bores, 352a and 354a, of cylindrical sections 352 and 354, and through central bore of entry port 110.

Referring now to FIG. 3D, which is a close-up on the inner elements of advancing mechanism 350. For the sake of clarity, the container enclosing the elements of advancing mechanism 350 is not shown in FIG. 3F. Applicator 353 is attached to wheel 357b via upper end of shaft 358b. Shaft 358b passes vertically via the axial center of wheel 357b and leaves the interior of advancing mechanism 350 via an aperture in sealing cover 351. The upper tip of shaft 358b is received inside a central bore in applicator 353. Wheel 357a is situated adjacent to wheel 357b, and the vertical shaft 358a passing through its axial center is parallel to shaft 358b. Canals 359 are formed on the outer circumferential surfaces of wheels 358a and 358b. Catheter tube 321 passes between wheels 357a and 357b such that portions of canals 359 tangential to catheter tube 321 are engaged with a portion of its outer surface.

Applicator 353 can be manually rotated by the practitioner, thereby applying a tangential force on portions of the outer surface of catheter tube 321 that are in contact with portions of canals 359, thus causing forward or backward movement of catheter tube 321, according to the direction of rotation. The advancing mechanism 350 is preferably designed such that only a certain amount of friction can be obtained between the portions of canals 359 being in contact with portions of the outer surface of catheter tube 321.

Limiting the friction between the contact surfaces of canals 359 and catheter 321 dictates a limitation on the amount of force that can be applied during the movement of the catheter tube 321. In this way, applying an excess amount of force will result in a slide of the wheels over the outer surface portion of catheter tube 321 being in contact with canals 359. As a result, the possibility of causing internal injuries to the treated subject by applying excess forces during insertion is substantially diminished.

FIG. 3E illustrates the catheter insertion device 300 in the inserted state. After completing the insertion of catheter tube 321, socket 230 receives into its interior the proximal cylindrical section 352 of advancing mechanism 350, and male fitting 238 (FIG. 3C) inside socket 230 is received in the respective female fitting provided in cylindrical section 352, thereby provide a sealed connection between the interior space of advancing mechanism 350 and the inner bore (not shown) of proximal hub 232. A sealed connection is obtained therebetween by engaging an inward inner protrusion 137 defined by the opening at the distal side of socket 230 with the annular groove 355 provided on the cylindrical section 352. After obtaining this sealed connection the sealing sleeve 320 can be removed by detaching clip 342 and thereby releasing the sleeve edge from the clip grasp, thereafter the requisite procedure can be performed.

The container of advancing mechanism 350 is preferably made from a type of polymer such as polypropylene, ABS, or polycarbonate, and it may be manufactured by injection molding techniques. Its height is generally in the range of 10 to 25 millimeters, and preferably about 15 millimeters, its vertical length is generally in the range of 15 to 35 millimeters, and preferably about 25 millimeters, and its width is generally in the range of 15 to 35 millimeters, and preferably about 20 millimeters. Cover 351 is preferably made from the same type of material and its sizes are determined according to the dimensions of container 350. Wheels 357a and 357b are preferably made from a type of polymer, such as polypropylene, Teflon, or ABS, and their diameters are generally in the range of 8 to 20 millimeters, and preferably about 12 millimeters. The vertical length of shaft 358 is determined according to the height of the container of mechanism 350. Shaft 358b is slightly prolonged in order to receive applicator at its upper end. The diameter of applicator 353 is generally in the range of 10 to 30 millimeters, and preferably about 18 millimeters.

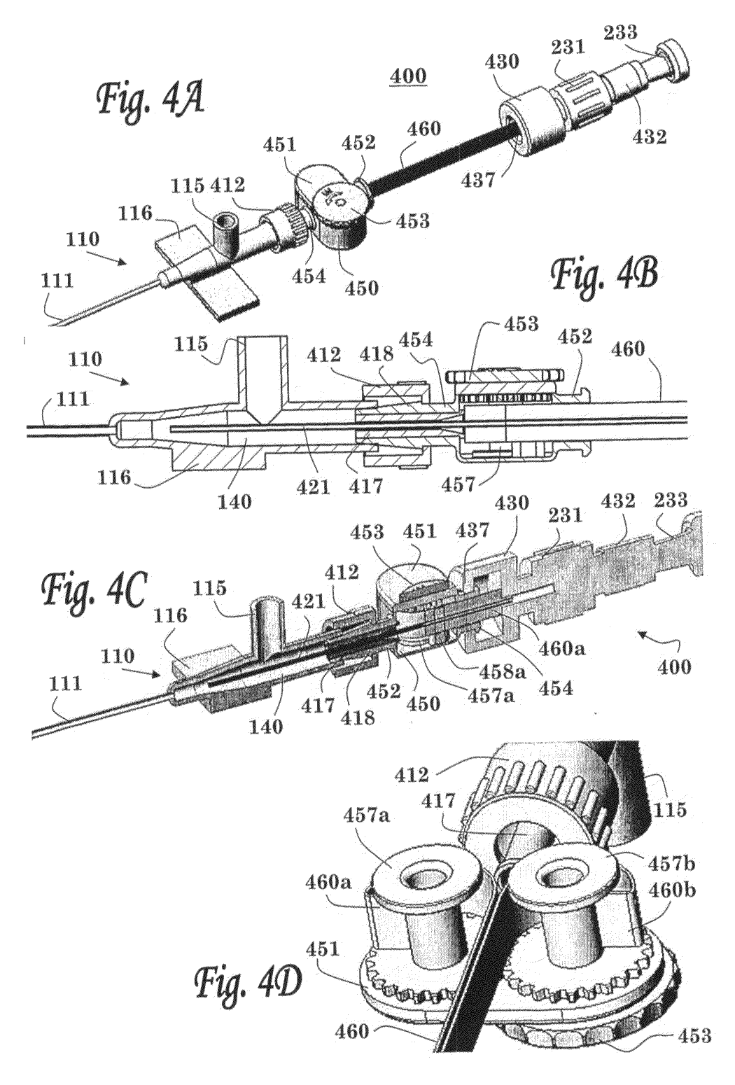

In another catheter insertion device of the invention a pair of adhered sealing strips 460 (hereinafter referred to as ‘sealing strip bilayer’) is used in catheter insertion device 400 for sealing and inserting the catheter tube 421, as shown in FIGS. 4A to 4E. The sealing strip bilayer 460 encloses catheter tube 421 which passes via the interior of proximal hub 432. The lumen of catheter tube 421 can be accessed via port 233. The sealing strip bilayer 460 is attached to socket 430 at its interior where an opening of an axial bore (not shown) in proximal hub 432 is provided. The sealing strip bilayer 460 is inserted into advancing mechanism 450 via a slender vertical passage provided along a cylindrical section 452 at the proximal end of advancing mechanism 450. In the advancing mechanism 450 the catheter tube 421 is separated from the sealing strip bilayer 460 and further advanced via an axial bore provided in a cylindrical section 454 at the distal end of advancing mechanism 450. Therefrom the catheter tube 421 is advanced into the central bore (140, FIG. 4C) of entry port 110, via an inner passage provided inside connector 412.

FIGS. 4D and 4E show the elements of the advancing mechanism 450, and other elements attached thereto. For the sake of clarity the container enclosing mechanism 450 is not shown in this FIG. 4D. The advancing mechanism 450 comprise two adjacently disposed winding wheels, 457a and 457b, each of which consists of a hollow cylindrical member for winding the respective strips 460a and 460b thereon, a gear wheel at one side of the cylindrical member and a guard wheel at the other side of said cylindrical member. The winding wheels 457a and 457b are attached at their guard wheel side by pivot protrusions 459 to the inner bottom side of the container such that their cylindrical members are substantially parallel. Similarly, winding wheel 457a is attached at the gear wheel side to the cover 451. Winding wheels 457a and 457b can freely rotate over pivot protrusions 459, and the teeth of their gear wheels are engaged such that rotation of one of said wheels is induced by the gear wheels to the other wheel. Shaft 455 attached to the axial center of winding wheel 457b passes via an aperture in cover 451 and received in a respective bore at the center of the applicator wheel 453.

Two adhered sealing strips 460a and 460b enclose the catheter tube 421 therewithin. The distal ends of said strips are attached to cylindrical members of the winding wheels 457a and 457b, respectively. In order to advance the catheter tube 421 the practitioner rotates (e.g., counterclockwise rotation in FIG. 4E) applicator wheel 453 thereby rotates the winding wheels 457a and 457b. In effect, the adhered strips 460a and 460b are separated and wound around the respective winding wheel, thereby exposing the catheter tube enclosed therein and advancing it toward passage tube 417.

Strips 460 may be adhered utilizing a high tack glue (i.e., having high degree of stickiness), but of a low strength, thereby enabling the operator to retract the portions of the inserted catheter tube and sealing it back inside strips 460 as it is pulled out. Such action may be required when the catheter is stuck and there is a need to retract a portion thereof in order to further advance it in a forward direction.

With reference to the longitudinal section view in FIG. 4B, where it is shown that passage tube 417 provides a passage for catheter tube 421 via an axial bore in cylindrical section 454. The proximal opening of passage tube 417 is conveniently of a conical shape for allowing easy insertion of the tip of catheter tube 421 thereto. The cylindrical section 452 comprises an annular groove adapted to receive an inward annular protrusion at the opening at the proximal side of connector 412. An inward annular protrusion is preferably also provided at the opening at the distal side of connector 412, which is adapted to fit over the outer surface of the proximal end of entry port 110. Cylindrical section 454 is followed by a conical member 418 the outer diameter of which is gradually decreased toward its distal end. The outer diameter of conical member 418 at the distal end is smaller than the diameter of the opening at the proximal end of entry port 110, which allows inserting it thereto in order to provide a sealed connection therebetween.

As shown in FIG. 4E, retaining members 458a and 458b are perpendicularly situated on the bottom side of the container of advancing mechanism 450 near the inner opening of the slender passage provided in cylindrical section 452. The sealing strip bilayer 460 passes between the retaining members 458a and 458b, which directs it towards the passage tube 417. For the sake of clarity, winding wheel 457a is not depicted in the advancing mechanism shown in FIG. 4F.

With reference to FIG. 4C, the cylindrical section 454 at the proximal side of the advancing mechanism 450 comprises an annular groove adapted to receive an inward annular protrusion 437 defined by the opening in socket 430. After completing the insertion of catheter tube 421 cylindrical section 454 is received inside socket 430 and a sealed connection therebetween is achieved by engaging inward annular protrusion 437 with the annular groove provided on cylindrical section 452, and the requisite procedure can be performed thereafter. Connector 412 preferably includes an inward annular protrusion at the opening at its distal side (not shown), which is adapted to fit over the outer surface of the proximal end of entry port 110.

Winding wheels 457 are preferably made from a type of polymer, such as polypropylene, polyethylene, or Teflon, and the diameter of their cylindrical members is generally in the range of 8 to 20 millimeters, and preferably about 12 millimeters. Strips 460a and 460b are preferably made from flexible, bacteria resistant with low “memory” type of material, such as paper or a non-woven fabric, and their width is generally in the range of 5 to 15 millimeters, and preferably about 8 millimeters. The strips 460a and 460b may be joined to seal catheter tube 421 therewithin by means of pressure stamping or low strength glue, preferably by utilizing an acrylic or silicone based glue.

The catheter insertion device which was described immediately hereinabove with reference to FIGS. 4A to 4D, can be modified in order to allow procedures to be performed with this device when partially inserted. FIG. 4E shows such a modification wherein vertical affixing canals 557 are provided on the lateral sides of the advancing mechanism 550. In this example, each canal 557 is formed by two adjacent parallel ribs formed on the lateral sides of advancing mechanism 550. Canals 557 are adapted to receive fastening arms 571 of affixing device 570. In this example fastening arms 571 are slightly elevated relative to the plane of the affixing device 570.

Affixing device 570 further comprise retaining arms 572 situated at the proximal end of said device. Retaining arms 572 protrude perpendicularly from the plane of affixing device 570 and adapted to receive an annular groove 439 formed between socket 430 and gripping section 231, thereby fastening proximal hub 432 thereto. The length of the strip between the advancing mechanism 550 and the socket 430 may vary (e.g., 20 to 60 millimeter). Since in the affixed state a predetermined distance is provide between the socket 230 and the advancing mechanism 550 (e.g., about 50 millimeters) supporting pins 575 situated at opposite sides of affixing device 570 and along its longitudinal length are provided for wrapping a portion of sealing strip bilayer 460 therebetween, whenever the length of the none-inserted strip bilayer is greater than said predetermined distance.

The advancing mechanism 550 used in this embodiment is substantially similar to the mechanism used in the embodiment previously described with reference to FIGS. 4A to 4D, and therefore it will not be discussed herein for the sake of brevity.

Affixing device 570 is preferably made from a type of polymer, such as polypropylene, ABS, or Polyethylene, its vertical length is generally in the range of 20 to 60 millimeters, and preferably about 50 millimeters, its width is generally in the range of 20 to 50 millimeters, and preferably about 30 millimeters.

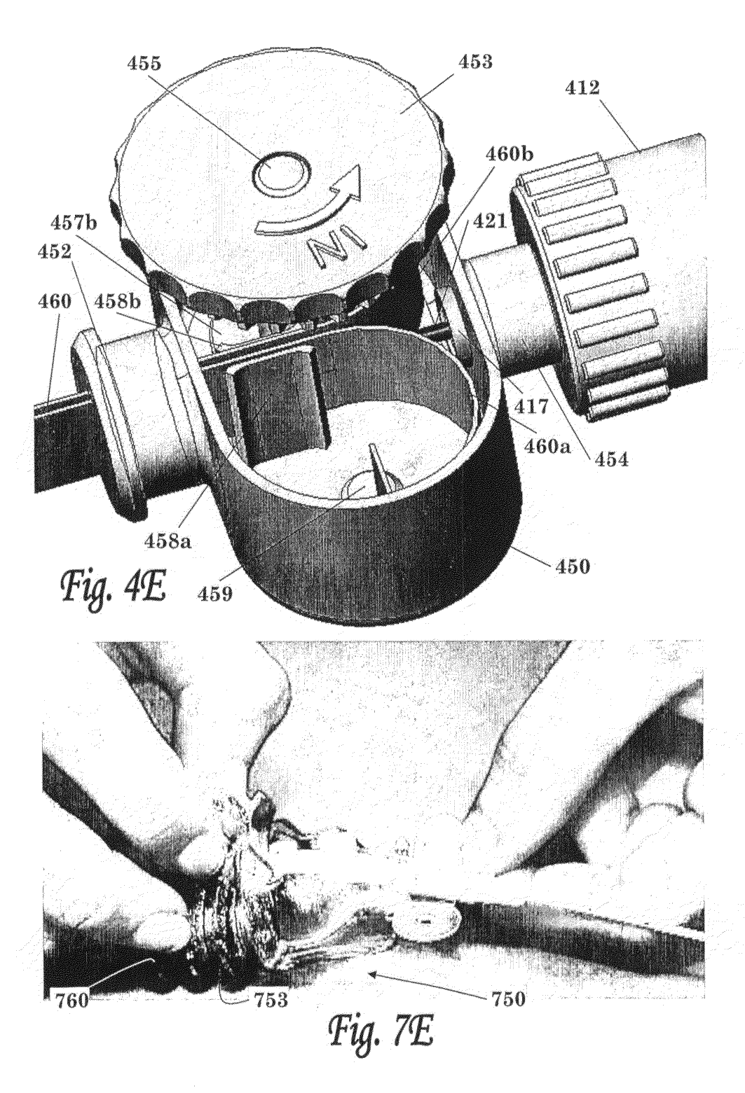

As was shown hereinabove, insertion of the catheter tube utilizing a sealing strip bilayer can be carried out without a sealing sleeve, and as was previously described, this preferred embodiment of the invention can be further utilized for carrying out procedures with a partially inserted catheter. An additional advantage of this insertion mechanism will be now discussed with reference to FIGS. 5A to 5D, which illustrates a catheter insertion device designed for allowing insertion of the catheter tube by a “pull” operation.

In this embodiment, shown in FIGS. 5A to 5D, the catheter insertion device 600 comprises a proximal hub 432, a sealing strip bilayer 460 containing catheter tube 421, strip separator apparatus 680, and entry port 110. Strip bilayer 460 is attached to the inner distal wall of socket 430 where an opening of a bore (not shown) in proximal hub 432 is provided. The catheter tube 421 sealed in strip bilayer 460 passes via said bore towards entry port 110. The distal portion of strip bilayer 460 is inserted into separator 680 via a slender passage passing via proximal adapter 682 of separator 680. This slender passage leads to the interior of separator 680, which further includes cover 685, retaining members 684, passage tube 683, splitters 686, distal adapter 652, and slender apertures 669a and 669b.

The retaining members, 684a and 684b, are perpendicularly disposed on the inner bottom side of the container of separator 680 adjacent to the inner opening of the slender passage at the proximal inner side of separator 680. The retaining members 684 define a slender passage therebetween which provides a passage for directing strip bilayer 680 towards the opening of passage tube 683 (FIG. 5B). The distance between the retaining members 684 is gradually increased toward the opening of passage tube 683, such that said tube can be received therebetween. Strip bilayer 460 is directed via the passage defined by retaining members, 684a and 684b, towards the opening of passage tube 683, disposed therein.

Strips 460a and 460b are separated near the opening of passage tube 683 and therefrom advanced separately towards respective slender apertures, 669a and 669b, along the lateral sides of splitters, 686a and 686b, respectively. Concurrently, Catheter tube 421 is exposed near the opening of passage tube 683, advanced into said tube towards an opening connecting the interior of said tube with an axial bore provided in the distal adapter 652 (FIG. 5C), and therefrom into the central bore 140 of entry port 110. The distal ends of strips 460a and 460b leave separator 680 via slender apertures 669, and their tips are joined together at 603, near hub 115.

In this state, shown in FIGS. 5A to 5C, the catheter insertion device 600 is ready for insertion, which is carried out by pulling the adhered strip tips at 603 forwardly towards the distal tip of insertion cannula 111. The catheter tube can be partially inserted and affixed utilizing an affixing device similar to that shown in FIG. 4E, or it may be fully inserted as shown in FIG. 5D.

When fully inserted, socket 430 is advanced until proximal adapter 682 is received via a distal aperture thereof into the hollow interior of socket 430. A sealed connection is obtained as the inward protrusion 437 at said aperture is engaged with an annular groove on the outer surface of adapter 682.

An inward annular protrusion is preferably provided at the opening at the distal side of connector 612 (not shown), which is adapted to fit over the outer surface of the proximal end of entry port 110. Distal adapter 652 comprise a conical member 618 adapted to be received in the proximal opening of entry port 110. In this example, the catheter insertion device 600, shown in FIGS. 5A-5C, includes a sealing sleeve 220 and a clip 642 for securing the sealing sleeve 220 to proximal adapter 682. However, it should be recognized that these elements are optional and in fact may be redundant due to the sealing of the catheter tube 421 that is provided by strip bilayer 460. For instance, the sealing sleeve 220 may be used as an extra defense for use when the glue used to join strip bilayer 460 is not suitable for retracting portions of catheter tube.

Separator 680 is preferably made from a type of polymer, such as polypropylene, ABS, or polycarbonate, and its vertical length is generally in the range of 20 to 50 millimeters, and preferably about 30 millimeters, its width is generally in the range of 10 to 30 millimeters, and preferably about 20 millimeters, and its height is generally in the range of 8 to 20 millimeters, and preferably about 15 millimeters.

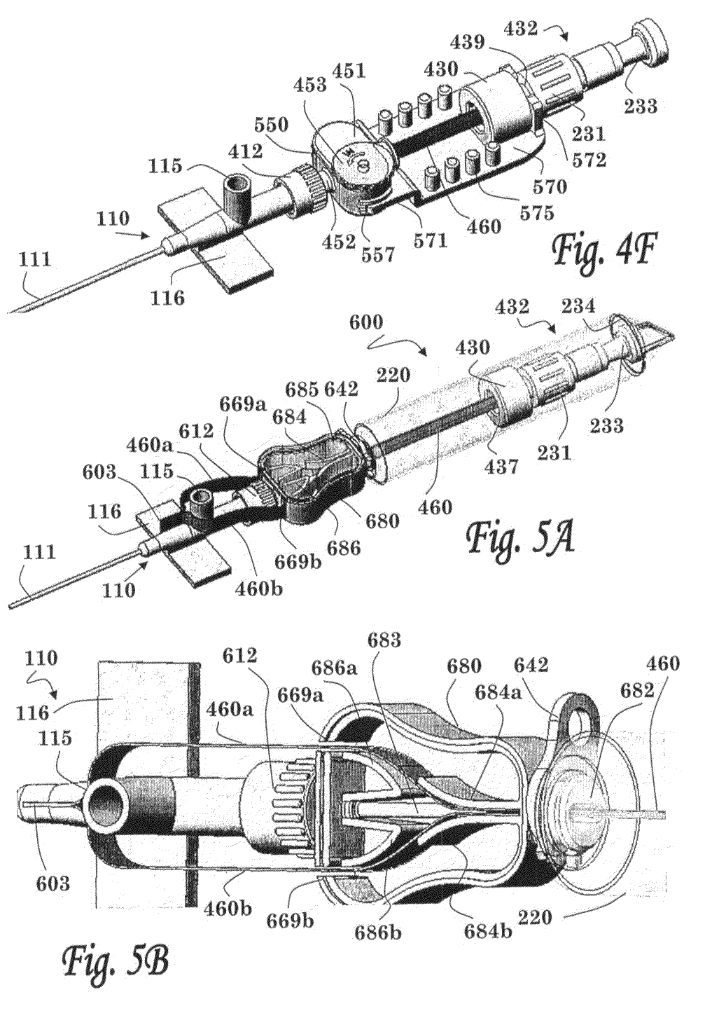

FIG. 6 demonstrates an advancing mechanism 700 wherein the catheter tube 721 is wound on a spool 701. Spool 701 comprises an axial cylindrical opening adapted to receive therein cylindrical member 702 which is provided in the center of sealed housing 710. The advancing mechanism 700 further comprises an exit tube 705 attached thereto from which the wound catheter tube 721 is rolled out. Connector 704 is attached to the exit tube 705 via a cylindrical member which is inserted into said tube. An axial bore (not shown) is provided inside connector 704, wherein the catheter tube 721 enter said bore at opening provided at the proximal end of the cylindrical member, and it leaves said bore at opening provided at the distal end of connector 704. Said distal end is adapted to provide a sealed connection with the proximal opening of an entry port in a way similar to the like connections which were previously discussed hereinabove. Catheter port 703 is attached to the proximal end of catheter tube 721 and provides access to its inner lumen for performing the requisite procedure.

The advancing mechanism 700 can be connected directly to an entry port 110, which was previously placed into a blood vessel, and the catheter tube 721 can be then advanced into said blood vessel via entry port 110 by rotating the spool 701 via a handle (not shown) connected to the inner side of cylindrical member 702, thereby allowing the operator to rotate spool 701. This advancing mechanism also permits performing procedures with a partially inserted catheter tube. The practitioner can carry out the requisite procedure via catheter port 703 at any suitable stage during the catheter insertion. The lid 707 of advancing mechanism 700 may be removed to allow access to the catheter port 703 at any stage during the insertion.

Advancing mechanism 700 is preferably made from a type of rigid polymer, such as polypropylene, polycarbonate, or ABS, its diameter is generally in the range of 30 to 60 millimeters, and preferably about 40 millimeters, its height is generally in the range of 8 to 20 millimeters, and preferably about 12 millimeters. The diameter of cylindrical member 702 is determined to fit in the cylindrical opening provide if spool 701.

The advancing mechanism 700 may be dismantled after insertion of the catheter tube 721, leaving a portion of the catheter tube 721 to be attached to the body of the treated patient. For example, exit tube 705 may include a longitudinal slit 708 through which catheter tube 721 may be removed. In this way after insertion of catheter tube 721 is completed connector 704 may pulled out of exit tube 705 and thereafter the housing 710 of advancing mechanism 700 may be discarded by removing the proximal portion of catheter tube 721 therefrom via an opening provided in housing 710 and via said slit 708.

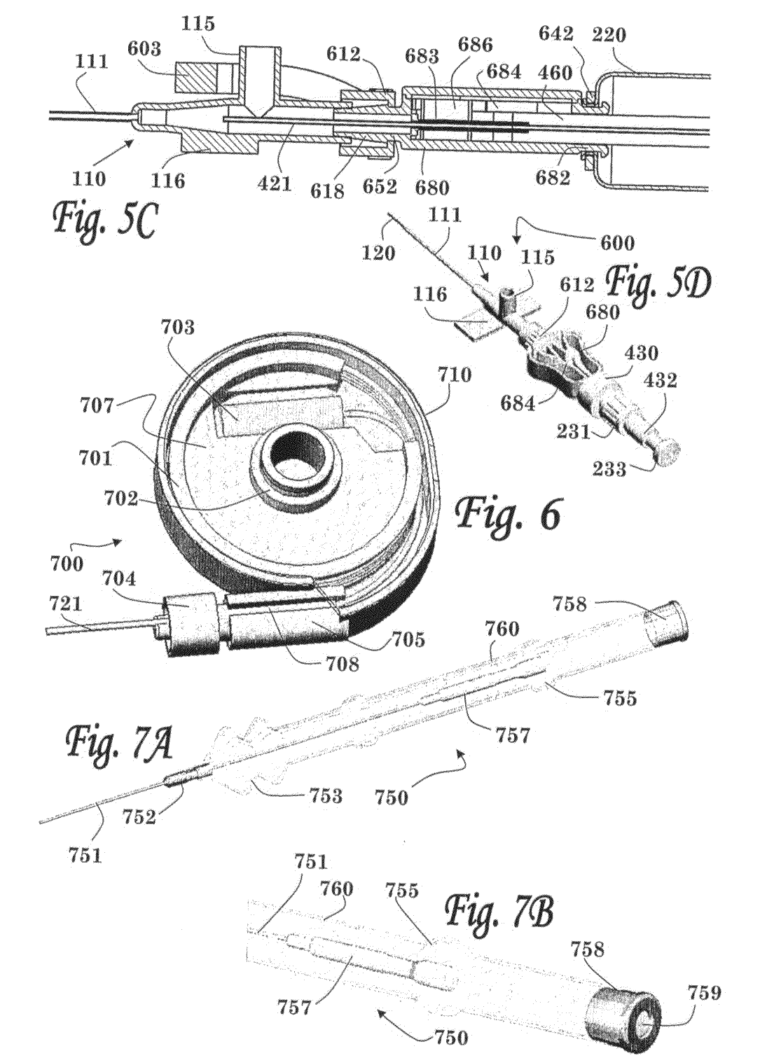

FIG. 7A is a perspective view of another catheter insertion device 750 of the invention which is made from a single flexible sleeve 760. While flexible sleeve may be permanently sealed at its proximal end, in the preferred embodiment illustrated in FIG. 7A cap 758 is sealably fitted into the proximal end opening of sleeve 760. The distal portion of sleeve 760 preferably comprises a tapering end in which a slender passage element 752 is sealably fitted. Slender passage 752 comprises a concentric bore (shown in FIG. 7D) suitable for passing catheter tube 751 therethrough.

The inner diameter of the concentric bore of slender passage element 752 is configured to provide close-fitting with the outer surface of catheter tube portion passing therethrough and thereby prevent passage of fluids (blood) therethrough into the interior of sleeve 760. In addition, slender passage element is preferably configured to produce sufficient traction for holding the catheter tube in place while flexible sleeve 760 moves back.

Catheter tube 751 is contained in catheter insertion apparatus 750 in a sterile and sealed environment defined by sleeve 760. Flexible sleeve 760 may be manufactured from a resilient material (e.g., silicon, polyurethane) to allow insertion of catheter tube 751 contained therein by grasping portions thereof by externally squeezing respective portions of sleeve 760 and pushing the same distally towards the slender passage element 752. In this way in each of these push actions portions of catheter tube 751 are advanced out of sleeve 760 via the concentric bore of slender passage element 752, and sleeve 760 restores its original shape when its grasped portions are released. In the preferred embodiment illustrated in FIG. 7A sleeve 760 comprises a resilient section 753 provided near the distal end of the sleeve 760 which conveniently enable the operator to carry out this advancing operation of catheter tube 751.

Flexible sleeve 760 may further comprise annular supports 755 provided along its length for increased stiffness and to help prevent kink and collapse of sleeve 760. Annular supports 755 may be implemented as annular portions of sleeve 760 wherein the said sleeve if thicker or by applying an external layer over the sleeve in various locations along its length. Alternatively, the entire sleeve 760 may be made thick to prevent kink and collapse thereof.

The distal portion of slender passage element 752 protrudes distally via a distal opening at the tapering distal end of sleeve 760 and it may comprise quick connection means (e.g., luer lock) for connecting catheter insertion device 750 to supplementary devices, such as entry port 110 (not shown) or a splittable introducer 761 (shown if FIG. 7D). For example, in FIGS. 7C and 7D, the distal portion of slender passage element 752 is fitted into an entry port of a splittable introducer 761 (e.g., PeelAway®). After completing the insertion of catheter tube 751 into the patient the splittable introducer 761 may be splitted and discarded by pulling tabs 768. In this state the entire length of catheter tube 751 is advanced into the patient and the tapering end of catheter port 757 (FIG. 7A) is fitted into cavity 771 (FIG. 7D) provided at the proximal end of slender passage element 752.

Cap 758 may comprise a concentric bore which at one end thereof opens into the interior of sleeve 760, and which other end is closed by a permeable material 759 (e.g., Tyvac, paper, polypropylene membrane) thus enabling the application of gas sterilization of the device's interior prior to carrying out any procedure therewith. Alternatively or additionally, permeable material portions may be provided at one or more locations on the lateral sides of sleeve 760.

With reference to FIGS. 7C and 7D, wherein a modified catheter insertion device 770 is illustrated which may be teared away. For this purpose tearing tab 766 is preferably provided at the distal end portion of sleeve 760, perpendicularly protruding therefrom between parallel tear lines 769 provided along the longitudinal length of sleeve 760. In this preferred embodiment the flexible sleeve 760 of the catheter insertion device 750 can be ripped and discarded, after completing the insertion of the catheter tube 751, by pulling upwardly tearing tab 766, as demonstrated in FIG. 7E.

It should be noted that slender passage element 752 can be made splittable in order to allow removal thereof after completing the catheter insertion. Furthermore, slender passage element 752 can be an integral part of flexible sleeve 760. Additionally, slender passage element 752 is preferably designed such that it will apply sufficient traction on the outer surface of the catheter tube portions passing thereinside in order to hold the catheter tube in place while the resilient section 753 restores its original shape. Optionally, slender passage element 752 may be configured as a one way valve which requires application of low forces in moving the catheter distally and relatively greater forces in retracting it proximally. It should be however clear that the catheter insertion device of the invention may be implemented without the slender passage element.

Flexible sleeve 760 may be manufactured by injection molding process from a resilient material such as silicon or polyurethane, its length is generally in the range of 10 to 300 mm, preferably about 50-mm, and its diameter is generally in the range of 6 to 30 mm, preferably about 18 mm. The diameter of slender passage element 752 is generally in the range of 0.5 to 3 mm, preferably about 1 mm, its length is generally in the range of 0.1 to 5 mm, preferably about 1 mm, and may be manufactured by injection molding from silicon, rubber, polyurethane, polyethylene. The diameter of cap 758 is generally in the range of 5 to 25 mm, preferably about 20 mm, its length is generally in the range of 5 to 20 mm, preferably about 15 mm, and it may be manufactured by injection molding from ABS, polypropylene. Tear lines 769 may be implemented by thin or weakened lines introduced during the manufacture process.

The resilient section 753 may be implemented by forming a corrugated section near the distal end of sleeve 760. Of course resilient section 753 may be also implemented in various other ways, for example by attaching a spring to the respective inner (or outer) surface of flexible sleeve 760.

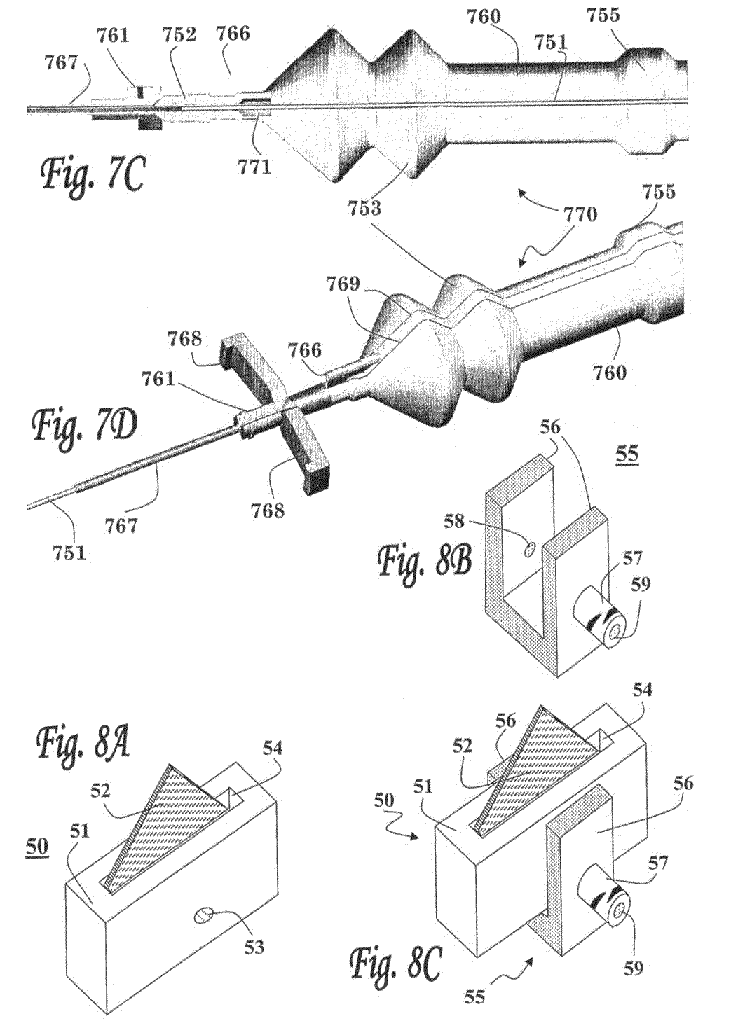

FIG. 8A schematically illustrates a conventional catheter cutter 50. This conventional cutter comprises a rectangular cutting blade 52 enclosed in housing 51. Portion of catheter tube may be trimmed by catheter cutter 50 by passing it (without its guide-wire) via apertures 53 provided on opposing lateral walls of housing 50, such that it is passed via the inner space of housing 50 and under the blade of cutting blade 52. After passing a certain portion of catheter tube for cutting via apertures 53 said portion may be cut off by pushing down the side of the cutting blade which protrudes outwardly via opening 54, thus pressing the blade of cutting blade 52 against the catheter tube passing in the inner space of housing 51 and cutting said portion off.

In order to allow carrying out such a trimming process with the catheter insertion device of the invention and the conventional catheter tube cutter 50 while maintaining a sterile environment the cutter adapter 55 shown in FIG. 8B may be used. Cutter adapter 55 is preferably made from a rigid material and formed in a “U”-like shape. The arms 56 of cutter adapter 55 are adapted to fit over the corresponding portions of the lateral walls of catheter tube cutter 50 in which apertures 53 resides, such that apertures 53 may be accessed via corresponding apertures 58 provided in arms 56 when the catheter tube cutter 50 is centered between arms 56 of cutter adapter 55, as shown in FIG. 8C.

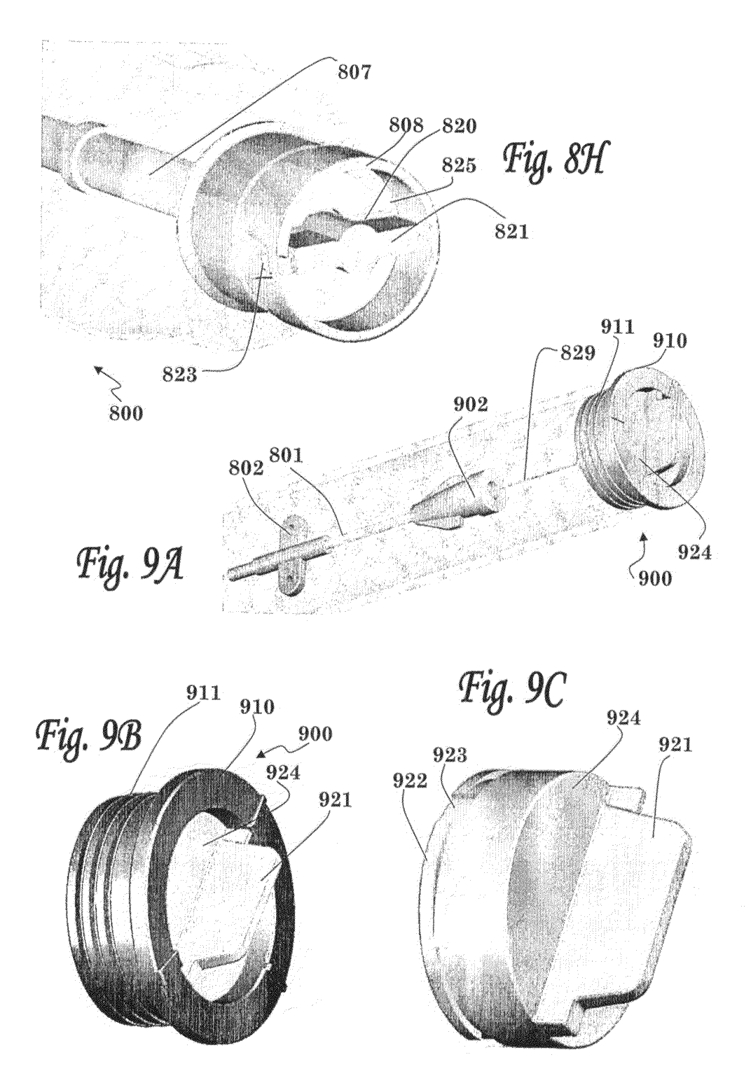

Connector 57 provided on the outer side of arms 56 provides a sealed access to apertures 58 via a concentric bore 59 provided therein, thereby allowing connecting cutter adapter 55 to a corresponding connectors means of the catheter insertion device of the invention (e.g., 943 in FIG. 10C) and advancing the catheter tube into the interior of catheter tube cutter 50 therethrough. It should be appreciated that the sealed connection obtained in this way between the catheter insertion device and the inner space of catheter tube cutter 50 provides the sterile environment required for carrying out catheter trimming.

Cutter adapter 55 can be made from a plastic or metal material, such as poly ethylene, poly propylene, ABS, Nylon, preferably from polypropylene, utilizing an injection molding manufacture process. The width of its arms 56, and of the side connecting the arms, is generally in the range of 5 to 20 mm, preferably about 12 mm, and their length is generally in the range of 10 to 20 mm, preferably about 15 mm. The distance between the arms should be set according to the width of the catheter tube cutter 50, for example it may range between 2 to 20 mm, preferably about 8 mm. The diameter of apertures 58 and concentric bore 59 should be set according to the diameter of apertures 53 of catheter tube cutter 50, for example it may range between 0.5 to 5 mm, preferably about 1 mm.

Cutter adapter 55 preferably comprise two connectors 57 each of which is attached on the outer surface of an arm 56 such that its concentric bores is in connection with the respective apertures 58, which allows sealing the passage provided between the concentric bores and via apertures 58 and 53 by a cap (not shown).

It should be clear that a sealed connection of the catheter insertion apparatus of the invention may be also obtained by utilizing a dedicated catheter tube cutter (not shown) which has one or two connectors 57 integrally attached to its sides over apertures 53 such that the interior of said dedicated catheter cutter may be accessed via concentric bores of the connectors. Of course, when such a dedicated catheter tube cutter is used cutter adapter 55 is not required.

Another catheter insertion device 800 of the invention is illustrated in FIG. 8D, wherein the insertion device 800 is implemented using a flexible sleeve 817 comprising a plurality of stiffening portions 816. Alternatively, flexible sleeve can be made rigid by increasing its thickness for preventing kink and collapse thereof. Catheter insertion device 800 may be manufactured from a resilient material and/or it may comprise a resilient (e.g., corrugated) portion 803. The catheter insertion procedure carried out using catheter insertion device 800 is substantially similar to the catheter insertion procedure that was previously described herein with reference to FIGS. 7A-7C.

In this example, sleeve 817 further comprises a tearing tab 811 and a slender passage element 812. The proximal section of catheter tube 801 contained in sleeve 817 comprises anchoring means 802 and catheter port 807. Guide wire 829 passing inside catheter tube 801 further comprises guide wire holder 821, which will be described in details hereinbelow.

Catheter insertion device 800 is connected via adapter 55′ to a catheter trimming device 50 which facilitates catheter trimming within a sterile environment, as may be required. FIG. 8E exemplifies the connection of the catheter insertion device of the invention 800 to conventional catheter cutter 50 via a suitable cutter adapter 55′. Such connection is preferably obtained by providing suitable quick connecting means (e.g., Luer lock) at the distal tip of the catheter insertion device and at the cutter adapter 55′ and thereby providing a sealed connection therebetween. Guide wire 829 (shown in FIG. 8G) may pass along the entire length of catheter tube 801 via its inner lumen or via a dedicated wire lumen (not shown). In this example the proximal end of guide wire 829 is attached to the sealed proximal end of sleeve 817 for preventing it from being advanced into the inner space of catheter tube cutter 50, thereby preventing the situation wherein its distal portion is cut.

Sleeve 817 may comprise permeable portions (not shown) for allowing applying gas sterilization procedures (e.g., Ethylene Oxide) to the interior of sleeve 817. Alternatively, one or all sides of the sleeve 817 may be manufactured from a permeable material (e.g., paper). It should be appreciated that this sleeve embodiment, which comprises permeable portions, may be advantageously used for production of catheter insertion kits that does not require the sterilization blister packaging that is commonly nowadays.

As seen in FIGS. 8D and 8E during the trimming process a proximal section 801a of the catheter tube 801 is advanced distally out of the catheter insertion device 800 for trimming. As shown in this example the catheter tube 801 is advanced distally via slender passage element 812, for example by using the resilient section 803 as was previously described hereinabove. While portions of catheter tube 801 are advanced distally, corresponding portions of guide wire 829 are withdrawn from catheter tube 801.

While attaching the proximal end of the guide wire to the proximal end of sleeve 817 may be sufficient to obtain the withdrawal of guide wire 829 during the advancing of catheter tube 801, it is also beneficial to have sleeve 817 made from a relatively rigid material (e.g., polypropylene), and/or of a sufficient thickness (e.g., 1.5 mm) to prevent displacements of guide wire 829 relative to sleeve 817. Alternatively, sleeve 817 may be packaged inside a rigid sleeve (not shown) that will prevent wrinkling and or collapse of sleeve 817 and thereby prevent displacements of guide wire 829 relative to sleeve 817.

The catheter portion 801a is trimmed by pushing the cutting blade down, after which the length of catheter tube 801 required for the specific procedure is obtained in the catheter insertion device 801. The procedure may then proceed by removing the catheter tube cutter 50 and cutter adapter assembly and connecting the catheter insertion apparatus 800 to a suitable introducer.

FIG. 8F is a close-up on the rear portion of the catheter insertion device shown in FIG. 8A which comprise a guide-wire stopper mechanism 808. In this example the proximal end of sleeve 817 terminates in a guide wire stopper 808 comprising a rigid partition 825 which close the proximal end of sleeve 817 while allowing it to hold guide wire holder 821 provided at the proximal end of guide wire 829. Aperture 820 may be provided in partition 825 through which guide wire holder 821 can be passed proximally out of sleeve 817. Aperture 820 and guide wire holder 821 can be configured in a plurality of shapes (e.g., curved, triangular, “X”, waved) for preventing distally longitudinal movements of guide wire 829 by rotating guide wire holder 821 about the longitudinal axis of guide wire 829 after passing it through aperture 820.

In the example shown in FIG. 8F the guide wire stopper 808 utilizes a rectangular aperture 820 and a corresponding rectangular guide wire holder 821 which provide the requisite proximal fixture of guide wire 829 by rotating (less than 180°) holder 821 about the axis of wire 829. This proximal fixture of guide wire 829 is conveniently established during catheter tube trimming for preventing cutting of the distal end portion or the wire 829. After completing the trimming process this proximal fixture can be released by rotating guide wire holder 821 until it is fitted into aperture 820 such that it may pass therethrough back into sleeve 817, as demonstrated in FIG. 8H.

Guide wire stopper 808 may be implemented by a hollow cylindrical element comprising a partition 825 thereinside which defines an external compartment 827 in which guide wire holder 821 can be fixtured, as demonstrated in FIG. 8F. Said external compartment 827 (FIG. 8G) may be sealed by a suitable cap 826 which may be fastened thereover by the aid of one or more flexible fasteners 823 provided on the external surface of stopper 808. Cap 826 may further contain a permeable section permeable to gases for allowing sterilization of the inner space of sleeve 817. During the trimming process portions of catheter tube are advanced distally out of sleeve 817 while respective portions of guide wire 829 are retracted from the catheter tube via catheter port 807.

Guide wire stopper 800 may be fabricated by injection molding techniques from rigid, polypropylene, ABS, polycarbonate type of material, preferably from ABS. The outer diameter of guide wire holder 800 is generally in the range of 6 to 30 mm, preferably about 20 mm, and its length is generally in the range of 5 to 30 mm, preferably about 20 mm.

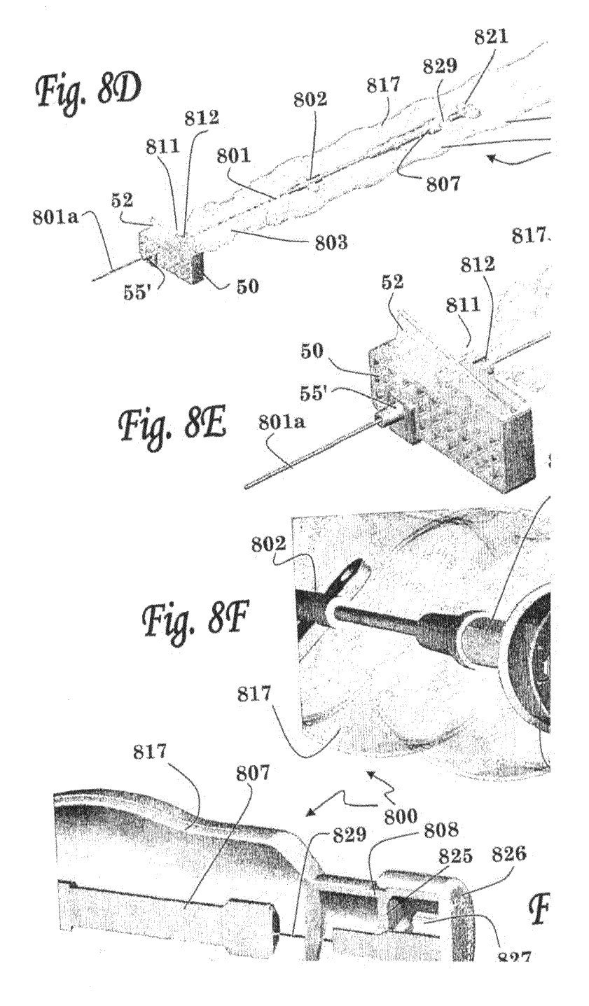

Another example for a guide wire stopper 900 is demonstrated in FIGS. 9A to 9E. In this example guide wire stopper 900 is designed to received a fastening cap 924 into an external compartment 925 provided therein. Fastening cap 924 is further configured to grip the guide holder (FIG. 9E) and thereby prevent accidental rotation and release thereof during the trimming process.

Guide wire stopper 900 may be implemented as a hollow cylindrical element having sealing threads 911 on the outer surface of a distal portion thereof adapted to be inserted into sleeve 901 of a catheter insertion device of the invention. Guide wire holder may further comprise a proximal flange 910 for preventing it from being pressed into sleeve 901. Sealing may have a hollow cylindrical shape which interior may be accessed via a distal opening. Fastening cap 924 further comprise an actuating tab 921 (FIG. 9C) and griping means provided in its hollow interior, for griping guide wire holder (not shown). An external groove 923 may be provided on the outer surface of fastening cap 924 near its distal rim 922 for locking it into guide wire stopper 900.

As seen in FIG. 9D, guide wire stopper 900 has a hollow cylindrical shape and its interior may be accessed via the proximal opening 927 or via aperture 920 provided in its distal side wall 925. Aperture 920 is configure to enable passage of a guide wire holder therethrough and fixture thereof by rotation, as was described herein above with reference to FIGS. 8F to 8H. Proximal opening 927 may be sealed by fastening cap 924 by fitting said cap into the internal space of guide wire stopper 900, and sliding inner protrusions 926 provided in stopper 900 into the external groove 923 of fastening cap 924.

FIG. 9E shows the interior of the fastening cap 924 as seen via opening 928 provided at its proximal side. Gripping means 929 provided in the hollow interior of fastening cap 924 are designed to enable gripping the guide wire holder when it is being held by guide wire stopper 900. Gripping means may be implemented by two or more opposing walls attached perpendicularly to the inner side of the base of fastening cap 924, as demonstrated in FIG. 9E.

Guide wire stopper 900 and fastening cap 924 may be manufactured by an injection molding process from a rigid material, preferably from ABS or polypropylrnr. The inner diameter of guide wire stopper 900 is generally in the range of 6 to 30 mm, preferably about 20 mm, and its length is generally in the range of 5 to 30 mm, preferably about 20 mm. The outer diameter of fastening cap 924 is configured to allow tight fitting it into guide wire stopper 900, its inner diameter is generally in the range of 5 to 25 mm, preferably about 10 mm, and its length is generally in the range of 5 to 25 mm, preferably about 15 mm.

A catheter insertion device 930 of the invention having a splittable head 932 is shown in FIG. 10A. Catheter insertion device 930 comprises a flexible sleeve 936 comprising a distal resilient portion 931 and which distal end is sealed by a splittable head 932. Flexible sleeve 936 may be manufactured from a single continuous sleeve, or alternatively, from two separate sleeves, 936a and 936b. For example, the catheter insertion device 930 may advantageously comprise a tearable sleeve section 936a and a relatively rigid sleeve section 936b, where the flexible sleeve 936 is assembled by fitting the proximal end of tearable sleeve 936a over the distal end of rigid sleeve 936b, as shown in FIG. 10A.

Catheter tube 801 comprised in sleeve 936 may be distally advanced out of catheter insertion device 930 via passage provided in the splittable head 932 by pushing it distally via resilient portion 931 of sleeve 936, as was previously described herein above with reference to FIGS. 7A to 7B. Resilient section 931 (e.g., corrugated section or containing shape restoring means such as a spring) is preferably provided near the distal end of catheter insertion device 930, however, the entire tearable sleeve section 936a may be advantageously manufactured from a resilient material (e.g., silicon). Tearable sleeve section 936a further comprise tear lines 935 for facilitating ripping thereof after the catheter insertion procedure in completed.

While tear lines 935 may be implemented in various ways (e.g., thin or weakened lines introduced during the manufacture process), in a preferred embodiment of the invention tear lines 935 are implemented by lines which are made on the tearable sleeve section 936a with a relatively decreased thickness.

Splittable head 932 comprise a cylindrical main body 933 having lateral pins 942 adapted to fasten splittable head 932 in the distal end of sleeve 936 by corresponding lateral apertures provided therein. Splittable head 932 further comprise splitting tabs 941 and a quick connector 943 (e.g., Luer lock) provided at its distal end portion. Quick connector 943 may be used to connect catheter inserter device 930 to a splittable introducer by means of a suitable adapter (not shown) for carrying our the catheter insertion process as was described hereinabove with reference to FIGS. 7A to 7D.

FIG. 10B shows a longitudinal section view of splittable head 932. In this example splittable head 932 is assembled from two connectable parts, 932a and 932b, each of which comprises a respective splitting tab 941, lateral pin 942, and cross-sectional portions of cylindrical main body 933 and quick connector 943. The hollow interiors of cylindrical main body 933 and of quick connector 943 are connector via slender passage element 937. Slender passage element 937 is configured to allow the passage of catheter tube 801 therethrough while preventing backflow of fluids such as blood into sleeve 936. Slender passage element 937 may be an integrated part of splittable head 932 such that each connectable part thereof, 932a and 932b, comprises a portion thereof, or alternatively, it may be a separate element attached to the inner wall of quick connector 943.

As demonstrated in FIGS. 10B to 10C, slender passage element 937 is made splittable in order to allow removal thereof after completing the catheter insertion. Additionally, slender passage element 937 is preferably designed such that it will apply sufficient traction on the outer surface of the catheter tube portions passing thereinside in order to hold the catheter tube in place while the resilient portion 931 restores its original shape. Optionally, slender passage element 937 may be configured as a one way valve which requires application of low forces in moving the catheter distally and relatively greater forces in retracting it proximally. It should be however clear that the catheter insertion device of the invention may be implemented without the slender passage element.

The connectable parts of splittable head 932 may be attached in various ways, for example by utilizing suitable glue or by welding. In a preferred embodiment of the invention splittable head 932 is assembled via coupling pegs 945 and corresponding peg-holes 947 which are provided in connectable parts 932a and 932b, as best seen in FIGS. 10B and 10C. After the insertion of the catheter tube 761 into the patient's body is completed the catheter insertion device 930 may be discarded by pulling apart splitting tabs 941 in opposite direction, or by pushing their tips proximally, and thereby splitting the coupled parts 932a and 932b of splittable head 932. After splitting splittable head 932 further pulling apart splitting tabs 941 will result in tearing of sleeve 936 along tear lines 935 provided thereon.

FIG. 10D schematically illustrates splitting splittable head 932 and tearing tearable sleeve section 936a, which is typically performed after completing insertion of the catheter tube 801. As demonstrated in FIG. 10D the splittable head 932 is splitted into two parts, 932a and 932b, which are pulled laterally in opposite directions. Pins 942 threaded into respective apertures provided in tearable sleeve section 936a initiate tearing of tearable sleeve section 936a along tear lines 935. Rigid sleeve portion 936b sealed by plug 938 remains intact and the proximal portion of the catheter 801 may be withdrawn and thereafter the catheter insertion device 930 may be disposed.

Rigid sleeve section 936b can be manufactured by extrusion from a semi-rigid type of material, preferably from polyethylene, its diameter is generally in the range of 10 to 35 mm, preferably about 20 mm, its length is generally in the range of 100 to 80 mm, preferably about 20 mm, and its thickness is preferably about 0.5 mm. Tearable sleeve section 936a can be manufactured by injection molding from a flexible type of material, preferably from silicon, its diameter is generally in the range of 5 to 30 mm, preferably about 15 mm, its length is generally in the range of 10 to 150 mm, preferably about 50 mm, and its thickness is preferably about 0.3 mm.

Splittable head 932 may manufactured by injection molding from a rigid type of material, preferably from polypropylene, its diameter is generally in the range of 8 to 25 mm, preferably about 15 mm, its length is generally in the range of 10 to 35 mm, preferably about 15 mm. Splitting tabs 941 are preferably an integral part of splittable head 932, their lateral length is generally in the range of 5 to 20 mm, preferably about 10 mm, and they may have a tapering shape as demonstrated in FIG. 10A.

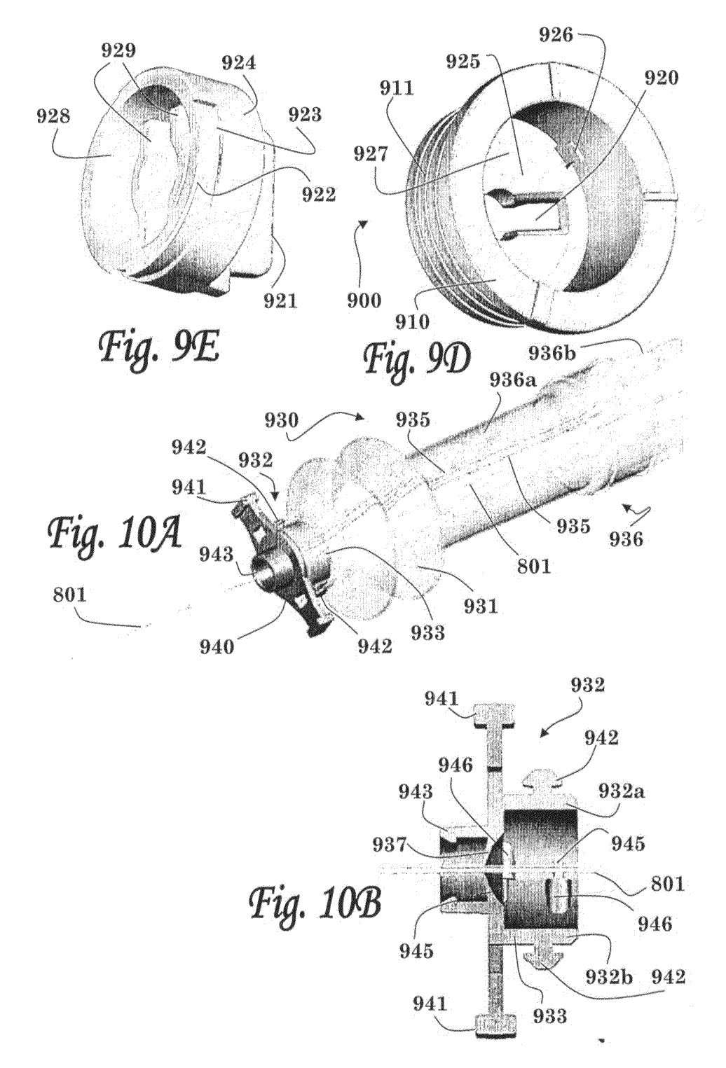

Another catheter insertion device 150 of the invention is schematically illustrated in FIG. 11A. In this catheter insertion device 150 a depressible element 152 is utilized for advancing catheter tube 162 passing inside elastic sleeve 160. Elastic sleeve 160 is sealed at its proximal end 153 and comprise a slender passage element 158 and an adapter 157 fitted in its distal end. The inner surface portion near the distal end of sleeve 160 is tightly fitted around the outer surface of adapter 157 and the tapering distal end of slender passage element 158 is tightly fitted into a concentric bore of adapter 157, thereby sealing the distal opening of sleeve 160 and providing an opening 156 through which catheter tube 162 may be advanced distally.

Sleeve 160 comprise an elevated section 159 which defines a forwarding tray on which the outer surface of catheter tube 162 may be grasped by the pushing edge of a depressible element 152, as seen in the side and top views in FIGS. 11A and 11B. Catheter tube 152 preferably comprises a guide wire 163 passing along its length via its lumen provided therein. Guide wire 163 is preferably attached to the distal end of sleeve 160 such that portions thereof are retracted from catheter tube 162 as portions thereof are distally advanced when trimming the catheter tube before inserting it into the vessel. Thereafter, the end of guide wire 163 must be released to enable insertion of the catheter into the vessel. Sealing cap 165 comprising a suitable connector may be fitted into adapter 157 for sealing the distal opening 156 of catheter insertion apparatus 150.

FIG. 11C schematically illustrates depressible member 152. Depressible member 152 consists of an inverted elongated “U” shaped member comprising arms 152d and a base section 152a connecting said arms 152d. An inverted “v” shaped member is attached to one of the lateral edges of base section 152a via a first “leg” 152b such that its second leg 152c extends downward away from said inverted elongated “U” shaped member. “U” shaped member is preferably made rigid while the “v” shaped member is made flexible to allow depressing it downward for pushing portions of catheter tube 162 distally via the free edge of leg 152c.

The apex of said “v” shaped member protrudes upwardly via an opening at the upper wall of sleeve 160 which is sealed by a flexible cupola shaped member 151 comprising said apex therein. Arms 152d are placed beside the proximal side of elevated section 159.

Sleeve 160 is preferably made from a transparent material, such as polyethylene, its length is generally in the range of 80 to 500 mm, preferably about 300 mm and its width is generally in the range of 8 to 30 mm, preferably about 22 mm. Slender passage element 158 is preferably made from a soft material, such as silicon, its length is generally in the range of 5 to 20 mm, preferably about 8 mm, the diameter of its tapering distal end is generally in the range of 2 to 10 mm, preferably about 4 mm, and the diameter of its inner bore is adjusted to allow passage of catheter tube therethrough while at least portions thereof are in contact with the outer surface of catheter tube, thereby sealing the interior of sleeve 160.

Adapter 157 is preferably made from a rigid material, such as polypropylene, its length is generally in the range of 8 to 30 mm, preferably about 15 mm, its outer diameter is generally in the range of 2 to 30 mm, preferably about 15 mm, and the diameter of its concentric bore is generally in the range of 3 to 10 mm, preferably about 4 mm.

Depressible element 152 may be manufactured from a flexible material, such as polypropylene, the length of its inverted elongated “U” shaped member is generally in the range of 5 to 80 mm, preferably about 10 mm, and the height of arms 152d is generally in the range of 5 to 50 mm, preferably about 20 mm. The length of the legs 152b and 152c of inverted “v” shaped member is generally in the range of 5 to 40 mm, preferably about 15 mm.

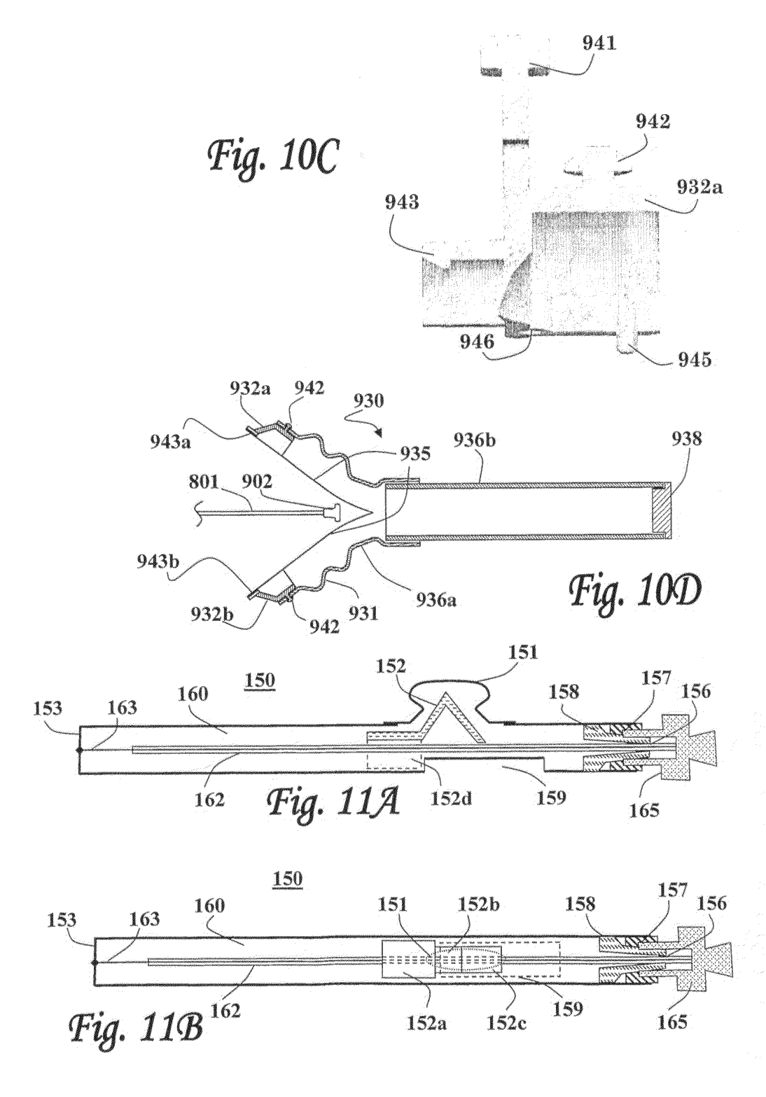

FIG. 12 schematically illustrates a catheter tube 162 comprising a slidable element 92 containing an antimicrobial substance. slidable element 92 is preferably situated near the distal tapering end of slender passage element 168, such that the outer surface of each portion of catheter 162 that is being advanced therethrough is treated and disinfected by said antimicrobial material. A further use of this antimicrobial agent is to disinfect the catheter as it moves in and out (micro-movements) after completing the insertion and when it is secured to the hand, this phenomenon is believed to be one of the causes for catheter infections. Antimicrobial substance, contained in slidable element 92 may be chlorhexidine, povidone Iodine or any other suitable disinfectant. The length of slidable element 92 is generally in the range of 1 to 20 mm, preferably about 5 mm, and it may be of cylindrical shape having a diameter in the range of 1 to 6 mm, preferably about 2 mm, and a concentric bore through which catheter tube 12 may pass. Slidable element 92 may of course be of any suitable shape allowing adhering it to the patient body while covering as much skin area as necessary. Slidable element 92 is preferably made from a soft material that can release the disinfectant contained therein over time and thereby keep the catheter's insertion area clean.

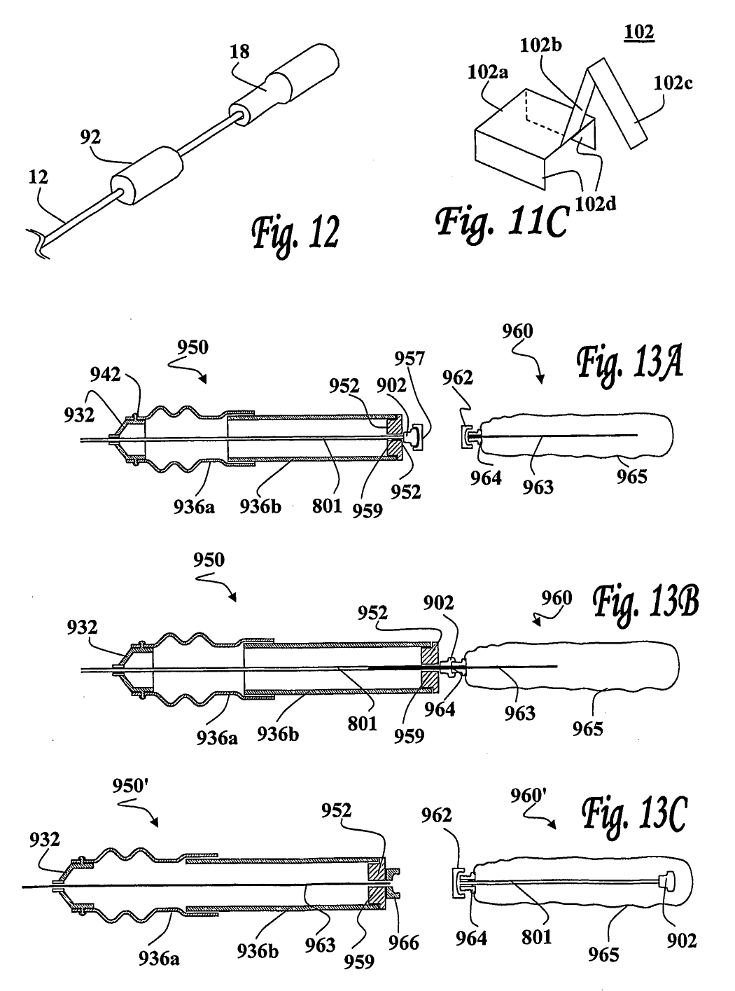

FIG. 13A schematically illustrates a catheter insertion device 950 of the invention containing catheter tube 801, wherein catheter port 902 passes via a tight aperture 959 provided in the plug 952 sealing the rear opening of rigid sleeve 936b. This configuration allows accessing the catheter lumen by removing sealing cap 957.

Also shown in FIG. 13A is a guide wire insertion device 960. Guide wire insertion device 960 comprises a sealed flexible sleeve 965 comprising guide wire 963 and having a distal exit port 964 holding a distal end portion of guide wire 963. Distal exit port 964 is configured to fit into catheter port 902 and thereby establish a sealed connection between the lumen of catheter tube 801 and the inner space of guide wire insertion device 960, as illustrated in FIG. 13B. This connection can be established by removing the sealing caps 962 and 975 and fitting the tip of distal exit port 964 into catheter port 902. In this state guide wire 963 may be advanced into the lumen of catheter tube 801 by grasping portions thereof via sleeve 965 and pushing guide wire 963 distally.

FIG. 13C demonstrates insertion devices 950′ and 960′ similar to those shown in FIGS. 13A and 13B, and which are configured to allow threading catheter tube 801 contained in insertion device 960′ over guide wire 963 contained in catheter insertion device 950′. In this example catheter insertion device 950′ is provided with hollow connector 966 configured to receive the tip of distal exit port 964 and thereby provide a sealed connection between the interior of catheter insertion device 950′ and of insertion device 960′.

Flexible sleeve 965 of insertion device 960′ may be manufactured by extrusion process from a transparent type of materials, preferably from polyethylene. The inner diameter of sleeve 965 is generally in the range of 2 to 20 mm, preferably about 6 mm, and its length is generally in the range of 100 to 600 mm, preferably about 300 mm. Distal exit port 964 may be manufactured by injection molding process from a rigid material, preferably from polypropylene and it may be sealably connected to the distal opening of flexible sleeve 965 by welding.

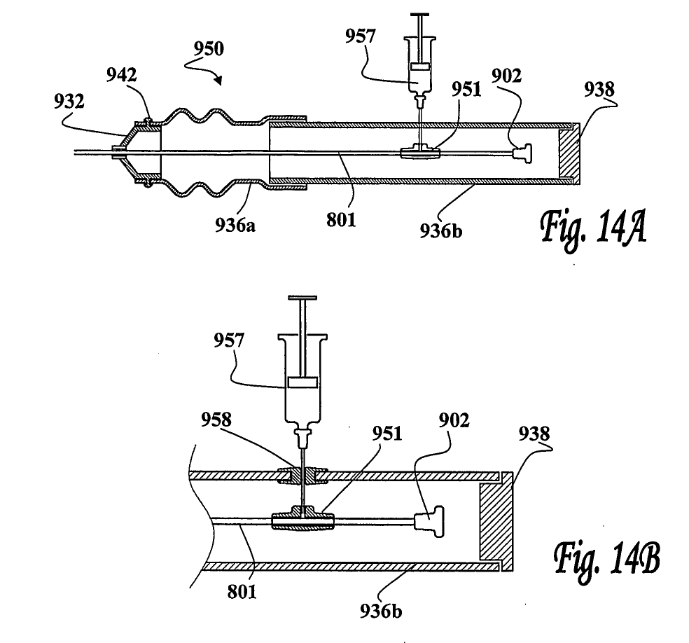

FIG. 14A schematically illustrates an embodiment of the catheter insertion device of the invention wherein the catheter tube 801 is provided with a lateral port 951. Lateral port is designed to provide access to the inner lumen of catheter tube 801 by a needle of syringe 957, for example, which may be required for washing said inner lumen of catheter tube 801. While the needle of syringe 957 may access lateral port 951 directly by puncturing sleeve 936b therewith, in another embodiment of the invention shown in FIG. 14B an access port 958 is provided for providing sealed access to lateral port 951.

Lateral port 951 may be manufactured by injection molding process from a sealable material, preferably from silicon. The inner diameter of lateral port 951 is generally in the range of 1 to 10 mm, preferably about 5 mm, and it may be applied over catheter tube 801 by welding. Access port 958 may be manufactured by injection molding process from a sealable material, preferably from silicon. The diameter of access port 958 is generally in the range of 2 to 15 mm, preferably about 6 mm, and it may be applied in a portion of the wall of sleeve 936b by tight fittings of materials.

All of the abovementioned parameters are given by way of example only, and may be changed in accordance with the differing requirements of the various embodiments of the present invention. Thus, the abovementioned parameters should not be construed as limiting the scope of the present invention in any way. In addition, it is to be appreciated that the different cylindrical/conical adapters, connectors, and other members, described hereinabove may be constructed in different shapes (e.g. having oval, square etc. form in plan view) and sizes from those exemplified in the preceding description.

The above examples and description have of course been provided only for the purpose of illustration, and are not intended to limit the invention in any way. As will be appreciated by the skilled person, the invention can be carried out in a great variety of ways, employing more than one technique from those described above, all without exceeding the scope of the invention.

1. A catheter insertion system comprising a flexible sleeve containing a catheter tube, wherein said flexible sleeve has a sealed proximal end, and wherein the distal end of said flexible sleeve comprises a slender passage adapted to allow passage of said catheter tube therethrough while preventing backflow of fluid into said sleeve.

2. The catheter insertion system according to claim 1, further comprising a resilient portion formed near the slender passage.

3. The catheter insertion system according to claim 2, wherein the resilient portion is corrugated.

4. The catheter insertion system according to claim 1, further comprising tear lines longitudinally passing along at least a portion of the length of the flexible sleeve, for facilitating tearing thereof.

5. The catheter insertion system according to claim 1, further comprising one or more permeable portions provided on the flexible sleeve for allowing gas sterilization of the interior of said flexible sleeve.

6. The catheter insertion system according to claim 1, wherein the proximal end of the flexible sleeve is sealed by a plug having a bore, wherein one end of said bore opens into the interior of the flexible sleeve, and wherein the other end of said bore is covered by a permeable sheet for allowing gas sterilization of the interior of said flexible sleeve.

7. The catheter insertion system according to claim 1, wherein the proximal end of the flexible sleeve is closed by a guide wire stopper element, wherein said guide wire stopper element comprises an aperture suitable for the passage of a guide wire holder connected to the proximal end of a guide wire, and wherein the passage of said guide wire holder into the flexible sleeve can be prevented by rotation of said guide wire holder about the axis of said guide wire.

8. The catheter insertion system according to claim 7, further comprising a fastening cap adapted to fit in or over the proximal side of the guide wire stopper element, thereby sealing the flexible sleeve, wherein said fastening cap comprises gripping means adapted to grip the guide wire holder and prevent passage thereof into said flexible sleeve.

9. The catheter insertion system according to claim 1, wherein the slender passage element is part of a splittable head sealably fitted into the distal end of the flexible sleeve, wherein said splittable head is constructed such that it can be longitudinally split into two or more parts, thereby facilitating tearing of the flexible sleeve.

10. The catheter insertion system according to claim 9, wherein the splittable head comprises pins adapted to be engaged in respective apertures provided in the flexible sleeve.

11. The catheter insertion system according to claim 9, further comprising splitting tabs provided on each splittable part of the splittable head.

12. The catheter insertion system according to claim 1, wherein the catheter tube comprises a lateral port adapted to allow access to the lumen of said catheter tube by a syringe needle passing through the wall of the flexible sleeve.

13. The catheter insertion system according to claim 12, wherein the flexible sleeve comprises a lateral port adapted to allow sealable access to the interior of said flexible sleeve by a syringe needle.

14. The catheter insertion system according to claim 1, wherein the proximal end of said catheter insertion device is sealed by a sealing plug comprising a slender passage through which the catheter tube is sealably passed such that its proximal port is external to said sleeve.

15. The catheter insertion system according to claim 1, further comprising a depressible element adapted for advancing the catheter tube contained in the flexible sleeve, wherein said depressible element comprises a flexible inverted “V” structure that is capable of advancing said catheter tube distally when pressed.

16. An adapter for a catheter tube cutter, comprising a hollow connector for connecting a catheter insertion device thereto, a base connected to said hollow connector, and an arm connected to said base opposite to said hollow connector, said arm comprises an aperture inline with an opening of said hollow connector, wherein a gap formed between said connector and said arm is suitable for fitting a catheter tube cutter therein and passing a catheter tube in the hollow interior of said connector via a cutter passage provided in said catheter cutter and said aperture in said arm.

17. A catheter insertion system comprising, at a first end, a distal entry port, at a second end a proximal section, a sealed catheter sleeve situated between said entry port and said proximal section, and further comprising advancing means for advancing a catheter contained within said system in a distal direction, wherein said entry port comprises a hollow body terminating at one end in a hollow cannula suitable for insertion into a peripheral blood vessel and having an internal diameter suitable for permitting passage of an intravascular catheter, and terminating at the other end with a connector element; wherein said proximal section comprises a hollow body having at least one external opening permitting the withdrawal and/or addition of fluid from or into the proximal opening of a catheter placed within said proximal section; and wherein said sealed catheter sleeve is sealably connected at one of its ends to either said entry port connector element or to advancing means located between said sleeve and said entry port connector element, said sealed catheter sleeve being sealably connected at its other end to said proximal section; such that when the aforementioned elements are connected together, a continuous hollow passageway exists from said proximal section through to the terminal portion of said hollow cannula, wherein said passageway contains the catheter that is to be inserted into a peripheral blood vessel.

18. The catheter insertion system according to claim 17, wherein the advancing means comprises the sealed catheter sleeve and a plurality of blades situated in close proximity to the distal end of said sleeve, such that when said sleeve is grasped and advanced in a distal direction, the catheter situated within said sleeve is similarly advanced, and said sleeve is progressively cut by said blades, thereby facilitating its removal from said system.

19. The catheter insertion system according to claim 17, wherein the advancing means comprises a flexible tube situated between the entry port and the sealed catheter sleeve, wherein said tube is adapted to permit an operator to grasp and compress said tube such that the catheter situated therewithin may be advanced in a distal direction.

20. The catheter insertion system according to claim 19, wherein the flexible tube contains corrugations along at least part of its length.

21. The catheter insertion system according to claim 17, wherein the advancing means comprises a rotating wheel mechanism situated between the entry port and the sealed catheter sleeve, such that rotation of an externally-situated thumb wheel causes rotation of one or more internally-situated wheels that cause the catheter to move in a distal or proximal direction.

22. The catheter insertion system according to claim 21, wherein the rotating wheels are adapted to provide a limited amount of friction between said wheels and the catheter tube, thereby limiting the amount of force applied on said catheter.

23. The catheter insertion system according to claim 21, wherein the sealed catheter sleeve consists of a pair of mutually adhered sealing strips, and wherein the rotating wheel mechanism further comprises a pair of winding wheels for separating and winding each of said sealing strips onto a spool.

24. The catheter insertion system according to claim 23, wherein the rotating wheel mechanism is adapted to retract portions of the inserted catheter tube and thereby to unwind portions of the sealing strips and seal the retracted portions therewithin.

25. The catheter insertion system according to claim 23, wherein an affixing device is utilized for affixing the proximal section of the system to the distal section for allowing performance of procedures with a partially inserted catheter.

26. The catheter insertion system according to claim 25, wherein the affixing device comprise supporting pins for wrapping a portion of the adhered sealing strips therebetween.

27. The catheter insertion, system according to claim 17, wherein the sealed catheter sleeve consists of a pair of mutually adhered sealing strips and wherein the advancing means comprises a sealed separator situated between the entry port and the sealed catheter sleeve, for separating the adhered sealing strips, exposing the catheter sealed therein, and advancing it into the entry port, and wherein said separator comprises a proximal aperture, splitting means, and retaining members for guiding said adhered strips, entering said separator via said proximal aperture, towards an opening of a passage tube linked to said entry port, and wherein the adhered strips are separated in the vicinity of said passage tube opening, advanced along the sides of said splitting means, and leave said separator via apertures provided thereon.

28. The catheter insertion system according to claim 27, wherein the tips of the separated strips are adhered outside the separator to allow convenient insertion of the catheter by pulling said adhered tips distally.

29. A catheter insertion system comprising, a distal entry-port and advancing means for advancing a catheter contained within said system in a distal direction, wherein said entry port comprises a hollow body terminating at one end in a hollow cannula suitable for insertion into a peripheral blood vessel and having an internal diameter suitable for permitting passage of an intravascular catheter, and terminating at the other end with a connector element; and wherein said advancing means comprises a sealed spool containing a sterilized catheter wound around a central axis in a spiral manner, and a distal exit, such that said catheter is rolled in or out via said distal exit by rotations of the spool and wherein the lumen of said catheter can be accessed via a catheter port connected to one end of the catheter wound for permitting the withdrawal and/or addition of fluid from or into the catheter.

30. The catheter insertion system according to claim 29, further comprising advancing means comprising a rotating wheel mechanism situated between the entry port and the sealed spool, such that rotation of said wheel causes rotation of one or more internally-situated wheels that cause the catheter to move in a distal or proximal direction, wherein the internally-situated wheels are preferably designed to provide a limited amount of friction between said wheels and said catheter tube, thereby limiting the amount of force applied on the thumb wheel.
